(19) United States
(12) Patent Application Publication
(10) Pub. No.: US 2005/0202559 A1
(43) Pub. Date: Sep. 15, 2005

(54) Cancer treatment by metabolic modulations

(76) Inventors: Scott Pownall, Vancouver (CA); Anthony T. Cheung, Vancouver (CA); Eric C. Hsu, Vancouver (CA); Bruno Doiron, Vancouver (CA)

Correspondence Address: Pillsbury Winthrop LLP;Intellectual Property Group, Suite 200, 11682 El Camino Real., San Diego, CA, 92130-2092, US

(21) Appl. No.: 10/697,700

(22) Filed: Oct. 29, 2003

Related U.S. Application Data
(60) Provisional application No. 60/422,365, filed on Oct. 29, 2002.

(51) Int. Cl. C12N 15/85; C12P 19/04

(52) U.S. Cl. 435/455; 435/101

(57) ABSTRACT

The invention provides compositions and methods for inhibiting the growth or proliferation of hyperproliferative cells or inducing regression of hyperproliferative cells. More specifically, the invention provides compositions and methods for stimulating glycogen accumulation in target cells (e.g., hyperproliferative cells) in order to increase glycogen to a level that is toxic to the target cell.

### RELATED APPLICATIONS

This application claims the benefit of priority of application Ser. No. 60/422,365, filed Oct. 29, 2002.

### FIELD OF THE INVENTION

The invention relates to inhibiting the growth or proliferation of hyperproliferative cells or inducing regression of hyperproliferative cells. More specifically, the invention relates to stimulating glycogen accumulation in target cells in order to increase glycogen to a level that is toxic to the target cell. The methods of achieving increased glycogen accumulation include, for example, increasing expression or activity of one or more genes that encode wildtype or mutant proteins (e.g., via gene transfer) that participates in glycogen synthesis or import and decreasing expression or activity of one or more genes that encode wildtype or mutant proteins (e.g., via antisense nucleic acid or small molecule) that participates in glycogen metabolism, catabolism, removal or degradation.

### BACKGROUND

Cancer is a leading cause of morbidity and mortality throughout the world. The magnitude of human and economic costs of cancer is enormous. In the United States alone, more than 1 million people are diagnosed with cancer each year and the total annual cost of cancer exceeds $870 billion, which constitutes approximately 4.7% of total annual healthcare spending. Although recent advances in early detection have led to an overall decline in cancer death rates, there is no universally effective strategy in preventing and treating cancer. The number of cancer cases is expected to rise in coming years due to a variety of reasons including ageing populations, environment pollution, etc.

Current cancer therapy generally depends on a combination of early detection and aggressive treatment involving surgery, chemotherapy, radiotherapy or hormone therapy. However, the invasiveness and generalized toxicity of such treatments present numerous deleterious side effects to the patients, thus seriously compromising their clinical effectiveness and patients' quality of life.

Furthermore, some cancer cells or tumors are inherently resistant to the cytotoxic drugs used in cancer treatment; others initially respond, but develop resistance during treatment as a result of selection pressure favoring the pre-existing resistant cell population and/or drug-induced mutations. Indeed, drug-resistance is a major cause of failure in cancer chemotherapy. It is also well recognized that radiotherapies are relatively ineffective in eradicating cancer cells within a solid tumor mass. Such failure is not surprising as radiotherapy requires free radicals derived from oxygen to destroy cells (Gray et al., Brit. J. Radiol. 26:683 (1953)), and oxygen levels inside a tumor mass are low due to the lack of proper blood supplies. Further, most chemotherapy drugs require oxygen for their efficacy (Giatromanolaki and Harris, Anticancer Res. 21:4317 (2001)). Therefore, there is a need for a cancer treatment that eliminates cancer cells and is able to exert its cytotoxic effects in a low oxygen or hypoxic condition.

The cause of cancer is still largely unknown. However, it is generally accepted that cancer formation or carcinogenesis is a complex process involving multiple genetic and environmental components. Given the incomplete understanding of the complex interplay between multiple carcinogenic factors, it is a formidable challenge to identify a therapeutic target that specifically and universally induce cancer cell death or inhibits tumor growth. With the advent of molecular biology and genetics, numerous signaling pathways that potentially contribute to the abnormal growth of cancer cells have recently been identified. For example, Ras is mutationally activated in about 30% of human cancers and overexpression of growth factor receptors (e.g. Epidermal Growth Factor, Insulin-like Growth Factor, Her2/Neu receptors) is commonly seen in different kinds of tumors. These observations have led to the discovery of chemical compounds design to block specific components in cancer-related signal transduction pathways. However, signal transduction in cancer cells involves highly divergent and redundant pathways and processes. Thus, the potential for resistance exists in the use of chemical drugs to block specific cellular pathways as a means to treat cancer.

Accordingly, there is a need for improved methods of treating cancer that provide an effective induction of cell death while minimizing side effects against normal cells. The invention addresses this need and provides related benefits.

### SUMMARY

The invention provides methods of increasing glycogen to toxic levels in a cell. An exemplary method includes expressing in the cell a gene product that increases the amount of glycogen to toxic levels in the cell. In various aspects, the gene product includes a protein that increases synthesis or intracellular accumulation of glycogen, for example, a glycogenic enzyme, or that decreases glycogen metabolism, catabolism, utilization, degradation or removal, for example, a glycogenolytic enzyme. In various additional aspects, the gene product that decreases glycogen metabolism, catabolism, utilization or degradation includes an inhibitory nucleic acid (e.g., antisense polynucleotide, a small interfering RNA molecule, or a ribozyme) of a glycogenolytic enzyme.

Target cells for practicing the methods of the invention include, for example, hyperproliferative cells, such as cells of a cell proliferative disorder; benign hyperplasia; and metastatic and non-metastatic tumors and cancer cells. Hyperproliferative cells appropriate for targeting can be in a subject, and in any organ or tissue. Exemplary organs and tissues include, for example, brain, head and neck, breast, esophagus, mouth, stomach, lung, gastrointestinal tract, liver, pancreas, kidney, adrenal gland, bladder, colon, rectum, prostate, uterus, cervix, ovary, testes, skin, muscle and the haematopoetic system.

Gene products useful in accordance with the invention include proteins, as well as inhibitory nucleic acid (e.g., antisense polynucleotide, a small interfering RNA molecule, or a ribozyme). Gene products can optionally be encoded by a polynucleotide, which can be included in a vector (e.g., a viral or mammalian expression vector). Gene products and polynucleotides can optionally be included in a vessicle. Expression of the polynucleotide can be driven by a regulatory element, such as a promoter active in a hyperproliferative cell (e.g., a hexokinase II, COX-2, alpha-fetoprotein, carcinoembryonic antigen, DE3/MUC1, prostate specific antigen, C-erB2/neu, telomerase reverse transcriptase or a hypoxia-responsive promoter).

Methods of the invention further include expressing in a target cell one or more additional gene products, optionally encoded by a polynucleotide. An exemplary gene product is a second protein that inhibits cell proliferation, such as a cell cycle inhibitor or a cyclin inhibitor.

The invention also provides methods of increasing glycogen to toxic levels in a hyperproliferative cell. An exemplary method includes contacting the cell with an agent that increases the amount of glycogen to toxic levels in the hyperproliferative cell. In one aspect, the hyperproliferative cell is not a liver, muscle or brain cell. In another aspect, the agent does not substantially inhibit activity or expression of a glycogen phosphorylase isotype (e.g., a liver, muscle or brain glycogen phosphorylase). In various additional aspects, the agent increases synthesis or intracellular accumulation of glycogen or decreases glycogen metabolism, catabolism, utilization, degradation or removal. In further aspects, the agent increases expression or activity of a glycogenic enzyme, or decreases expression or activity of a glycogenolytic enzyme. Exemplary agents include substrate analogues. Additional exemplary agents include inhibitory nucleic acids (e.g., antisense polynucleotide, a small interfering RNA molecule, or a ribozyme) that decrease or inhibit glycogen metabolism, catabolism, utilization or degradation.

The invention methods that increase glycogen to toxic levels optionally include one or more morphological changes associated with glycogen toxicity, such as cell swelling, increased numbers of lysosomes, increased size of lysosomes, or a structural change in lysosomes. Increasing glycogen to toxic levels also includes methods that cause lysis or apoptosis of the cell, or that inhibits or reduces proliferation, growth or survival of the cell.

Exemplary glycogenic enzymes useful in accordance with the invention, and whose expression or activity can be stimulated or increased include, for example, glycogenin, glycogenin-2, glycogen synthase, glycogenin interacting protein (GNIP), protein phosphatase 1 (PP-1), glucose transporter (GLUT), a glycogen targeting subunit of PP-1 isoform or family member, a hexokinase isoform or family member, and glutamine-fructose-6-phosphate transaminase. Exemplary glycogen targeting subunit of PP-1 family members include G_{L} (PPP1R3B, PPP1R4), PTG (PPP1R3C, PPP1R5), PPP1R3D (PPP1R6) or Gₘ/R_{G1} (PPP1R3A, PPP1R3).

Exemplary glycogenolytic enzymes useful in accordance with the invention, and whose expression or activity can be inhibited or decreased include, for example, glycogen phosphorylase, debranching enzyme, phosphorylase kinase, glucose-6-phosphatase, PPP1R1A (protein phosphatase 1, regulatory Inhibitor subunit 1A), PPP1R2 (protein phosphatase 1, regulatory subunit 2), phosphofructokinase, a glycogen synthase kinase-3 isoform, GCKR glucokinase regulatory protein and α-glucosidase.

The invention further provides methods of treating a cell proliferative disorder in a subject. An exemplary method includes expressing in one or more cells comprising the disorder a gene product that increases the amount of intracellular glycogen, sufficient to treat the cell proliferative disorder. Another exemplary method includes contacting one or more cells comprising the disorder with an agent that increases the amount of intracellular glycogen, sufficient to treat the cell proliferative disorder. In one aspect, the cell proliferative disorder is not a liver, muscle or brain cell disorder. In another aspect, the agent does not substantially inhibit activity or expression of a glycogen phosphorylase isotype (e.g., a liver, muscle or brain glycogen phosphorylase).

Cells proliferative disorders for practicing the methods of the invention include, for example, benign hyperplasia, metastatic and non-metastatic tumors and cancers. Tumor and cancer cells can be in a subject, and in any organ or tissue. Exemplary organs and tissues include, for example, brain, head and neck, breast, esophagus, mouth, stomach, lung, gastrointestinal tract, liver, pancreas, kidney, adrenal gland, bladder, colon, rectum, prostate, uterus, cervix, ovary, testes, skin, muscle and the haematopoetic system. Tumors and cancers can be solid or liquid, in any stage, such as a stage I, II, III, IV or V tumor, or be in remission. Exemplary tumor types include, for example, sarcomas, carcinomas, melanomas, myelomas, blastomas, gliomas, lymphomas and leukemias.

The invention moreover provides methods of treating a subject having a tumor. An exemplary method includes expressing in one or more of the tumor cells a gene product that increases the amount of intracellular glycogen, effective to treat the subject. Another exemplary method includes contacting one or more of the tumor cells an agent that increases the amount of intracellular glycogen, effective to treat the subject. In one aspect, the tumor is not a liver, muscle or brain tumor. In another aspect, the agent does not substantially inhibit activity or expression of a glycogen phosphorylase isotype (e.g., a liver, muscle or brain glycogen phosphorylase).

Methods of treatment include prophylactic methods as well as methods in combination with another treatment protocol. Thus, where a subject has a cell proliferative disorder, such as a tumor, for example, the subject can be treated before diagnosis or symptoms of the tumor appear, while the subject is undergoing a tumor therapy or after the subject has undergone tumor treatment, e.g., when the tumor is in remission. Accordingly, the gene product or agent can be administered prior to, substantially contemporaneously with or following administration of another therapy, e.g., an anti-tumor or immune-enhancing therapy.

Administration in accordance with a method of the invention can result in increasing effectiveness of another therapy. For example, administering a subject that is undergoing or has undergone anti-tumor or immune-enhancing therapy can increase the amount of intracellular glycogen, thereby increasing effectiveness of an anti-tumor or immune-enhancing therapy. In one aspect, the tumor therapy is not for a liver, muscle or brain tumor. In another aspect, the agent does not substantially inhibit activity or expression of a glycogen phosphorylase isotype (e.g., a liver, muscle or brain glycogen phosphorylase). Thus, methods of treatment include administering one or more additional therapies. Exemplary therapies include, for example, administering an anti-tumor or immune enhancing treatment or agent.

The invention additionally provides methods of treating a subject, which result in an improvement of the subject's condition, e.g., a reduction of one or more adverse symptoms of a cell proliferative disorder. For a tumor, for example, an exemplary method of treatment reduces tumor volume, inhibits an increase in tumor volume, inhibits progression of the tumor, stimulates tumor cell lysis or apoptosis, inhibits tumor metastasis, or prolongs lifespan of the subject.

Exemplary subjects for practicing the invention include mammals, such as humans, which include subjects having or at risk of having a cell proliferative disorder. Subjects further include, for example, are candidates for cell proliferative disorder therapy, or that are undergoing, or have undergone such therapy. For a tumor, for example, exemplary treatments include anti-tumor and immune-enhancing therapy.

Exemplary anti-tumor therapies include, for example, chemotherapy, immunotherapy, surgical resection, radiotherapy or hyperthermia. Exemplary anti-tumor therapies further include, for example, treatment with an anti-tumor agent such as an alkylating agent, anti-metabolite, plant extract, plant alkaloid, nitrosourea, hormone, nucleoside or nucleotide analogue, more particularly, cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin or dibromomannitol.

Exemplary immune enhancing treatment include, for example, administration of a lymphocyte, plasma cell, macrophage, dendritic cell, NK cell or B-cell. Exemplary immune enhancing treatments further include, for example, treatment with an immune enhancing agent such as a cell growth factor, survival factor, differentiative factor, cytokine or chemokine, more particularly, IL-2, IL-1α, IL-1β, IL-3, IL-6, IL-7, granulocyte-macrophage-colony stimulating factor (GMCSF), IFN-γ, IL-12, TNF-α, TNFβ, MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROβ, GROβ, ENA-78, GCP-2, PBP/CTAPIIIβ-TG/NAP-2, Mig, PBSF/SDF-1, or lymphotactin.

The invention provides cell-free and cell-based methods of identifying agents having anti-cell proliferative activity. An exemplary method includes: contacting a cell that produces glycogen with a test agent; and assaying for glycogen toxicity in the presence of the test agent or following contacting with the test agent. Glycogen toxicity identifies the test agent as an agent having anti-cell proliferative activity. Another exemplary method includes: contacting a cell that expresses a glycogenic enzyme or a glycogenolytic enzyme with a test agent; and measuring activity or expression of the glycogenic enzyme or glycogenolytic enzyme in the presence of the test agent or following contacting with the test agent. Increased or decreased expression or activity of the glycogenic enzyme or glycogenolytic enzyme, respectively, identifies the test agent as an agent having anti-cell proliferative activity. A further exemplary method includes: contacting a cell that expresses a gene whose expression is controlled by a regulatory region of a glycogenic enzyme or a glycogenolytic enzyme with a test agent; and measuring expression of the gene in the presence of the test agent or following contacting with the test agent. Increased or decreased expression of the gene identifies the test agent as an agent having anti-cell proliferative activity.

Yet another exemplary method includes: providing a test agent that modulates (increases or decreases) expression or activity of a glycogenic or a glycogenolytic enzyme; contacting a cell that expresses a glycogenic or a glycogenolytic enzyme with the test agent; and assaying for glycogen toxicity in the presence of the test agent or following contacting with the test agent. Glycogen toxicity identifies the test agent as an agent having anti-cell proliferative activity. Still another exemplary method includes: contacting a glycogenic enzyme or a glycogenolytic enzyme with a test agent; and measuring activity of the glycogenic enzyme or glycogenolytic enzyme in the presence of the test agent or following contacting with the test agent. Increased or decreased activity of the glycogenic enzyme or glycogenolytic enzyme, respectively, identifies the test agent as an agent having anti-cell proliferative activity.

Methods of identifying agents having anti-cell proliferative activity can employ assaying for glycogen toxicity, which can be determined, for example, by screening for a morphological change associated with glycogen toxicity, screening for cell viability, screening for inhibition or reduction of cell proliferation, growth or survival.

Methods of identifying agents can employ assaying for changes in gene expression or activity (e.g., a glycogenic or a glycogenolytic enzyme or a reporter). Exemplary glycogenic and glycogenolytic enzymes, as well as reporters are as set forth herein.

Methods of identifying agents can be performed in solution, in solid phase, in vitro, or in vivo.

Cells that can be screened or otherwise employed in the invention methods as targets are prokaryotic or eukaryotic. The cells can be stably or transiently transformed with a nucleic acid sequence (e.g., gene) whose expression is controlled by a regulatory region (e.g., of a glycogenic or glycogenolytic enzyme. The cells include hyperproliferative cells, immortalized cells, and tumor and cancer cells.

The invention provides kits. An exemplary kit includes an amount of an agent that increases expression or activity of a glycogenic enzyme, and instructions for administering the agent to a subject in need of treatment on a label or packaging insert. Another exemplary kit includes an amount of an agent that decreases expression or activity of a glycogenolytic enzyme, and instructions for administering the agent to a subject in need of treatment on a label or packaging insert. Yet another exemplary kit includes an amount of an agent that increases accumulation of intracellular glycogen, and instructions for administering the agent to a subject in need of treatment on a label or packaging insert. Kits optionally further include an anti-tumor or immune enhancing agent, pharmaceutical formulations, and articles of manufacture for delivering the agent into a subject locally, regionally or systemically, for example.

### DESCRIPTION OF DRAWINGS

FIGS. 1A and 1B show reduced HeLa cell viability and increased glycogen deposition after infection with AdG_{L}, which is time and viral vbector dose dependent. (A) HeLa cells infected with 200 MOI (light gray bars) or 1000 MOI (dark grey bars) adenovirus for the times indicated. Each bar represents the percentage of viable cells from AdG_{L}-infected cells compared to control AdpSh infected cells expressed as a percentage. (B) HeLa cells infected with 200 MOI or 1000 MOI adenovirus as indicated. Intracellular glycogen levels after vector transduction were assayed at times indicated. Bars represent glucose derived from glucoamylase-reduced glycogen in cells infected with AdG_{L} and cells infected with AdpSh (pSh). Higher viral vector doses result in higher glycogen levels.

FIGS. 2A to 2D show reduced cell viability and increased glycogen accumulation after infection of a human colorectal cancer (LoVo) and a human breast cancer cell line (MCF7), with AdG_{L}. (A) LoVo cell viability following infection at 100 MOI with AdG_{L} as compared to AdpSh. (B) Increased accumulation of glucose derived from glucoamylase-reduced glycogen in LoVo cells resulted from increased MOI of AdG_{L} compared to control AdpSh. MCF7 cells show (C) reduced viability and (D) increased accumulation of glucose derived from glucoamylase-reduced glycogen when infected with 45 MOI AdG_{L} compared to 45 MOI control AdpSh.

FIGS. 3A and 3B show that AdG_{L} in combination with cell cycle inhibitor roscovitine increases glycogen levels and further reduces cell viability in comparison to AdG_{L} alone. (A) AdG_{L} and roscovitine (black bars) significantly increased glucose derived from glucoamylase-reduced glycogen in infected HeLa cells over time in comparison to either AdG_{L} alone (dark grey bars) or roscovitine in combination with control AdpSh (medium grey bars). (B) Roscovitine significantly decreased cell viability of AdG_{L}-infected cells. The ratio of viable cells from AdG_{L}-infected cells compared to that of control AdpSh-infected cells is expressed as a percentage for both roscovitine-treated (grey bars) and untreated cells (white bars). All treatments were with 100 MOI of virus.

FIGS. 4 shows that genetic elements can increase expression of G_{L} in order to further reduce cancer cell viability. The four viruses used were AdpSh with no G_{L}, AdG_{L} with G_{L} but no enhancing element, AdhspGL with G_{L} and the hsp70 5′ UTR element, and AdG_{L} WPRE with G_{L} with and the WPRE element. All viruses were used at 100 MOI. Ratio of viable cells from virus-infected cells to control AdpSh-infected cells expressed as a percentage.

### DETAILED DESCRIPTION

The invention provides methods of modulating levels of intracellular glycogen. By modulating intracellular levels of glycogen, cells can alternately be relieved of glycogen or accumulate glycogen. Glycogen accumulation in cells can be toxic which can lead to an inhibition or a decrease in cell proliferation, growth, survival or viability. When glycogen accumulates at sufficient levels to produce toxicity, cell death can result. Thus, undesirable cell proliferation, as well as abnormal and diseased hyperproliferating cells (e.g., cell proliferative disorders such as tumors and cancer cells) can be targeted in order to reduce proliferation, growth, survival or viability of the target cells.

Glycogen can be induced or stimulated to accumulate in cells by a variety of mechanisms. For example, expression or activity of an enzyme that directly or indirectly participates in glycogen synthesis, production or accumulation, referred to herein as a “glycogenic enzyme,” can be induced or increased thereby increasing intracellular amounts of glycogen. In another example, expression or activity of an enzyme that directly or indirectly participates in glycogen metabolism, catabolism, utilization, degradation or removal, referred to herein as a “glycogenolytic enzyme,” can be inhibited or decreased thereby increasing intracellular amounts of glycogen. Although several proteins that participate in glycogen synthesis, production or accumulation, or glycogen metabolism, catabolism, utilization, degradation or removal are not technically enzymes since they do not catalyze a substrate to product reaction, for example, GLUT is a glucose transporter and glycogen targeting subunit family are adaptor molecules that associate PP-1 with glycogen, for convenience, such proteins are also termed glycogenic and glycogenolytic enzymes as used herein due to their participation in the various pathways that modulate glycogen levels. The invention therefore includes methods of increasing intracellular levels of glycogen regardless of the particular physiological or biochemical mechanism.

Modulating expression or activity of an enzyme that participates in glycogen synthesis, production, accumulation, metabolism, catabolism, utilization, degradation, or removal can be achieved by a variety of methods. For example, one or more glycogenic enzymes, or a gene encoding a glycogenic enzyme, can be introduced into a cell in order to increase levels of intracellular glycogen. In another example, an inhibitory nucleic acid (e.g., antisense, ribozyme, small interfering RNA or triplex forming polynucleotide) or a nucleic acid encoding an inhibitory nucleic acid can be introduced into a cell in order to increase levels of intracellular glycogen. An inhibitory nucleic acid sequence that targets a glycogenolytic enzyme, or encodes antisense that targets glycogenolytic enzyme, can be introduced into a cell in order to increase levels of intracellular glycogen. Intracytoplasmic introduction of appropriate nucleic acid or protein can stimulate or induce intracellular glycogen accumulation, optionally to toxic levels.

Thus, in accordance with the invention, there are provided methods of modulating (increasing or decreasing) intracellular glycogen, optionally to toxic levels in a cell (e.g., a hyperproliferative cell). In one embodiment, a method of increasing glycogen includes expressing in the cell a gene product that increases the amount of glycogen, optionally to toxic levels in the cell. In various aspects, the gene product is a protein that increases synthesis or intracellular accumulation of glycogen, or a protein that decreases glycogen metabolism, catabolism, utilization degradation or removal. In particular aspects, the gene product comprises a glycogenic enzyme (e.g., encoded by a polynucleotide), or an antisense polynucleotide, a small interfering RNA molecule, or a ribozyme that targets a glycogenolytic enzyme.

Specific non-limiting examples of glycogenic enzymes include: glycogenin, glycogenin-2, glycogen synthase, glycogenin interacting protein (GNIP), protein phosphatase-1 (PP-1), a glycogen targeting subunit of PP-1 isoform or family member, a hexokinase isoform or family member, or glutamine-fructose-6-phosphate transaminase. Glycogen targeting subunit of PP-1 isoforms and family members include G_{L} (PPP1R3B, PPP1R4), PTG (PPP1R3C, PPP1R5), PPP1R3D (PPP1R6) or Gₘ/R_{G1} (PPP1R3A, PPP1R3). A particular example of a glycogenic enzyme that indirectly participates in glycogen accumulation, is a glucose transporter (GLUT), which transports glucose into cells for glycogen synthesis. Exemplary glycogenic enzyme names (using the Hugo nomenclature), sequences and corresponding Genbank accession numbers include:

Glycogenin

GYG glycogenin NM_{—}004130

GYG2 glycogenin 2 NM_{—}003918

Glycogenin Interacting Protein (GNIP)

AF396651, AF396655, AF396654

Protein phosphatase-1

PPP1CA protein phosphatase 1, catalytic subunit, alpha isoform NM_{—}002708

Glycogen Synthase

GYS1 glycogen synthase 1 (muscle) NM_{—}002103

GYS2 glycogen synthase 2 (liver) NM_{—}021957

Glucose Transporters

SLC2A1 solute carrier family 2 (facilitated glucose transporter), member 1 NM_{—}006516 GLUT1

SLC2A2 solute carrier family 2 (facilitated glucose transporter), member 2 NM_{—}000340 GLUT2

SLC2A3 solute carrier family 2 (facilitated glucose transporter), member 3 NM_{—}006931 GLUT3

SLC2A4 solute carrier family 2 (facilitated glucose transporter), member 4 NM_{—}001042 GLUT4

SLC2A6 solute carrier family 2 (facilitated glucose transporter), member 6 NM_{—}017585 GLUT9 ,GLUT6

SLC2A7 solute carrier family 2 (facilitated glucose transporter), member 7 AL356306

SLC2A8 solute carrier family 2 (facilitated glucose transporter), member 8 NM_{—}014580 GLUTX1, GLUT8

SLC2A9 solute carrier family 2 (facilitated glucose transporter), member 9 NM_{—}020041 Glut9, GLUTX

SLC2A10 solute carrier family 2 (facilitated glucose transporter), member 10 NM_{—}030777 GLUT10

SLC2A11 solute carrier family 2 (facilitated glucose transporter), member 11 NM_{—}030807 GLUT11, GLUT10

SLC2A12 solute carrier family 2 (facilitated glucose transporter), member 12 NM_{—}145176 GLUT12, GLUT8

SLC2A13 solute carrier family 2 (facilitated glucose transporter), member 13 NM_{—}052885 HMIT

SLC2A14 solute carrier family 2 (facilitated glucose transporter), member 14 NM_{—}153449 GLUT14

Hexokinase Isoforms and Family Members

GCK glucokinase (hexokinase 4, maturity onset diabetes of the young 2) NM_{—}000162

HK1 hexokinase 1 NM_{—}033500

HK2 hexokinase 2 NM_{—}000189

HK3 hexokinase 3 (white cell) NM_{—}002115

Glutamine-fructose-6-phosphate Transaminase

GFPT1 glutamine-fructose-6-phosphate transaminase 1 NM_{—}002056

GFPT2 glutamine-fructose-6-phosphate transaminase 2 NM_{—}005110

Glycogen Targeting Subunit of PP-1 Isoforms and Family Members

PPP1 R3B protein phosphatase 1, regulatory (inhibitor) subunit 3B NM_{—}024607 G_{L}, FLJ14005, PPP1R4

PPP1 R3C protein phosphatase 1, regulatory (inhibitor) subunit 3C NM_{—}005398

PPP1R5, PTG

PPP1R3D protein phosphatase 1, regulatory subunit 3D NM_{—}006242 PPP1R6

PPP1 R3A protein phosphatase 1, regulatory (inhibitor) subunit 3A (glycogen and sarcoplasmic reticulum binding subunit, skeletal muscle) NM_{—}002711 PPP1R3, Gₘ/R_{G1}

Specific non-limiting examples of glycogenolytic enzymes include:

glycogen phosphorylase, debranching enzyme, phosphorylase kinase, glucose-6-phosphatase, PPP1R1A (protein phosphatase 1, regulatory Inhibitor subunit 1A), PPP1R2 (protein phosphatase 1, regulatory subunit 2), phosphofructokinase, a glycogen synthase kinase-3 isoform, GCKR glucokinase regulatory protein, or α-glucosidase. Exemplary glycogenolytic enzyme names (using the Hugo nomenclature), sequences and corresponding Genbank accession numbers include:

Glycogen Phosphorylase

PYGB phosphorylase, glycogen; brain NM_{—}002862

PYGL phosphorylase, glycogen; liver (Hers disease, glycogen storage disease type VI) NM_{—}002863

PYGM phosphorylase, glycogen; muscle (McArdle syndrome, glycogen storage disease type V) NM_{—}005609

Phosphorylase Kinase

PHKA1 phosphorylase kinase, alpha 1 (muscle) NM_{—}002637

PHKA2 phosphorylase kinase, alpha 2 (liver) NM_{—}000292

PHKB phosphorylase kinase, beta NM_{—}000293

PHKG1 phosphorylase kinase, gamma 1 (muscle) NM_{—}006213

PHKG2 phosphorylase kinase, gamma 2 (testis) NM_{—}000294

PHKGL phosphorylase kinase, gamma-like

CALM1 calmodulin 1 (phosphorylase kinase, delta) NM_{—}006888

CALM2 calmodulin 2 (phosphorylase kinase, delta) NM_{—}001743

CALM3 calmodulin 3 (phosphorylase kinase, delta) NM_{—}005184

Glycogen Synthase Kinase-3

GSK3A glycogen synthase kinase 3 alpha NM_{—}019884

GSK3B glycogen synthase kinase 3 beta NM_{—}002093

Glucose-6-phosphatase

G6PC glucose6-phosphatase, catalytic (glycogen storage disease type I, von Gierke disease) NM_{—}000151

Protein Phosphatase 1, Regulatory Subunit

PPP1R1A (protein phosphatase 1, regulatory (inhibitor) subunit 1A),

PPP1 R2 (protein phosphatase 1, regulatory subunit 2),

Phosphofructokinase

PFKL phosphofructokinase, liver NM_{—}002626

PFKM phosphofructokinase, muscle NM_{—}000289

PFKP phosphofructokinase, platelet NM_{—}002627

Glucosidase

AGL amylo-1,6-glucosidase, 4-alpha-glucanotransferase (glycogen debranching enzyme, glycogen storage disease type II) NM_{—}000646

GAA glucosidase, alpha; acid (Pompe disease, glycogen storage disease type II) NM_{—}000152

GANAB glucosidase, alpha; neutral AB

GANC glucosidase, alpha; neutral C AF545045

MGAM maltase-glucoamylase (alpha-glucosidase) NM_{—}004668

GCKR glucokinase (hexokinase 4) regulatory protein NM_{—}001486

Expression or activity of an enzyme that participates in glycogen synthesis, production, accumulation, metabolism, catabolism, utilization, degradation or removal can also be modulated by agents or treatments. Such agents or treatments can act directly or indirectly upon the proteins that participate in glycogen synthesis, production, accumulation, metabolism, catabolism, utilization, degradation, or removal.

For example, substrate analogues of glycogenolytic enzymes that are either poorly modified or not modified by the enzyme are a particular example of such an agent class. Substrate analogues may bind to the active site of the enzyme and either inhibit or prevent binding of a natural substrate, thereby increasing glycogen levels. Sugar and carbohydrate analogues (e.g., pseudooligosaccharides) are a particular example of a class of agents useful for inhibiting or reducing expression or activity of a glycogenolytic enzyme. Substrate analogues also include polypeptides and mimetics that mimic the naturally occurring substrate. For example, GSK-3 phosphorylates glycogen synthase which in turn inactivates the enzyme thereby reducing levels of glycogen. Thus, an analogue of glycogen synthase is one particular examples of an agent that inhibits GSK-3.

Thus, in accordance with the invention, there are also provided methods of modulating glycogen in a cell using an agent that increases the amount of intracellular glycogen. In one embodiment, a method includes contacting a cell (e.g., a hyperproliferative cell) with an agent that increases the amount of glycogen to toxic levels, wherein the cell is not a liver, muscle or brain cell. In another embodiment, a method includes contacting a cell with an agent that increases the amount of glycogen to toxic levels, provided that the agent does not substantially inhibit activity or expression of a glycogen phosphorylase isotype (e.g., a liver, muscle or brain glycogen phosphorylase isotype). In one aspect, the agent increases or stimulates expression or activity of a glycogenic enzyme. In another aspect, the agent reduces or inhibits expression or activity of a glycogenolytic enzyme. In additional aspects, the hyperproliferative cell comprises a benign hyperplasia or a metastatic or non-metastatic cancer cell. The cancer cell may be in culture (in vitro) or in vivo, for example, in brain, head or neck, breast, esophagus, mouth, stomach, lung, gastrointestinal tract, liver, pancreas, kidney, adrenal gland, bladder, colon, rectum, prostate, uterus, cervix, ovary, testes, skin, muscle or hematopoetic system, of a subject.

As used herein, the terms “substantial” and “substantially,” when used in reference to whether an agent or treatment “inhibits, reduces, increases or stimulates” expression or activity of a particular enzyme, such as a glycogen phosphorylase isotype, is a provision meaning that the agent or treatment does not affect activity of that particular enzyme (e.g., glycogen phosphorylase) to increase intracellular glycogen to toxic levels in cells. For example, an agent that does not substantially inhibit a glycogen phosphorylase isotype does not inhibit the enzyme at the agent concentration used to the extent that intracellular glycogen accumulates to toxic levels. There are three known human glycogen phosphorylase isotypes present in liver, muscle and brain. Thus, to “substantially inhibit” these glycogen phosphorylase isotypes means that enzyme activity is reduced or inhibited enough to increase intracellular glycogen to levels that are toxic (e.g., reduced cell proliferation, growth, survival, viability, etc.) in liver, muscle or brain.

Agents and treatments that act indirectly to stimulate or inhibit a glycogenic or glycogenolytic enzyme, e.g., a glycogen phosphorylase isotype, for example, inhibiting an intermediary protein which in turn inhibits glycogen phosphorylase activity, are not excluded by this provision. Agents and treatments that directly target or bind a glycogenic or glycogenolytic enzyme, such as glycogen phosphorylase, and at the concentration used increase intracellular glycogen to less than toxic levels (e.g., the amount of agent used is less than that needed to kill the cell) also are not excluded by this provision. Accordingly, this provision, when used, refers to agents and treatments, including the specific non-limiting examples of agents and treatments set forth herein, that target or bind to a glycogenic or glycogenolytic enzyme such as glycogen phosphorylase, and whose effect is to increase intracellular glycogen to toxic levels at the concentration of the agent or treatment used.

Agents include small molecules. As used herein, the term “small molecule” refers to a molecule that is less than about 5 kilodaltons in size. Typically, such small molecules are organic, but can be an inorganic molecule such as an element or an ionic form, for example, lithium, zinc, etc.

Specific non-limiting examples of agents that reduce or inhibit expression or activity of a glycogenolytic enzyme include glycogen phosphorylase inhibitors such as N-methyl-beta-glucose-C-carboxamide (Watson et al., Biochemistry, 33:5745 (1994)), Alpha-D-glucose (Oikonomakos et al., Eur. J. Drug Metab. Pharmacokinet. 19:185 (1994)), Glucopyranosylidene-spiro-hydantoin 16 (Somsak et al., Curr. Pharm. Des. 15:1177 (2003)), N-acetlyl-N′-β-D-glucopyranosyl urea (Acurea) and N-benzoly-N′-β-D-glucopyranosyl urea (Bzurea) (Oikonomakos et al., Eur. J. Biochem. 269:1684 (2002)), N-acetyl-beta-D-glucopyranosylamine (Board M., Biochem. J. 328:695 (1997)), Phenacyl imidazolium (Van Schaftingen and De Hoffmann E Eur. J. Biochem. 218:745 (1993)), CP-91149 (an indole-2-carboxamide) (Latsis et al., Biochem. J. 368:309 (2002)), Flavopiridol (Kaiser et al., Arch. Biochem. Biophys. 386:179 (2001)), Inole-2-carboxamides (Hoover et al., J. Med. Chem. 41:2934 (1998)), S-3-isopropyl-4-(2-chlorophenyl)-1,4-dihydro-1-ethyl-2-methyl-pyridine-3,5,6-tricarbo xylate (W1807) (Oikonomakos et al., Protein Sci. 10:1930 (1999)), BAY R3401 and BAY W1807 (Bergans et al., Diabetes, 49:1419 (2000); Shiota et al., Am. J. Physiol. 273:E868 (1997)), 1,4-dideoxy-1,4-imino-d-arabinitol (DAB) (Fosgerau et al., Arch. Biochem. Biophys. 380:274 (2000)), 5-chloro-1H-inodole-2-carboxylic acid (1-(4-fluorobenzyl)-2-(4-hydroxypiperidin-1-yl)-2-oxoethyl)amide (CP320626) (Oikonomakos et al., Structure, 8: 575 (2000)), Pyridoxal (5′)diphospho(1)-alpha-D-glucose (Withers G. J. Biol. Chem. 260:841 (1985)), 3,4-Dichloroisocoumarin (3,4-DC) (Rusbridge and Beynon FEBS Lett. 268:133 (1990)), caffeine (San Juan Serrano et al., Int. J. Biochem. Cell. Biol. 27:911 (1995)), alpha-, beta-, and gamma-cylodextrins (Pinotsis et al., Protein Sci. 12:1914 (2003)), glucopyranosylidene spirothiohydantoin (Oikonomakos et al., Bioorg. Med. Chem. 10:261 (2002)), aminoguanidine (Sugita et al., Am. J. Physiol. Endocrinol. Metab. 282:E386 (2002)), proglycosyn (Yamanouchi et al., Arch. Biochem. Biophys. 294:609 (1992)), and 2-deoxy-2-fluoro-α-D-glucopyranosyl fluoride (Massillon et al., J. Biol. Chem. 270:19351 (1995)).

Additional non-limiting examples of agents that reduce or inhibit expression or activity of a glycogenolytic enzyme include glycogen synthase kinase-3 isoform (α or β) inhibitors. Inactivation of glycogen synthase kinase 3 (GSK-3) leads to the dephosphorylation of substrates including glycogen synthase and eukaryotic protein synthesis initiation factor-2B (eIF-2B). This results in their functional activation thereby increasing intracellular glycogen.

Small molecule inhibitors of GSK-3 include drugs such as hymenialdisine (e.g., Dibromo-hymenialdisine) (Breton and Chabot-Fletcher, J. Pharmacol. Exp. Ther. 282:459 (1997); Meijer, et al., Chem. Biol. 7:51 (2000)); indirubins (e.g., 5,5′-dibromo-indirubin) (Damiens et al., Oncogene 20:3786 (2001); Leclerc et al., J. Biol. Chem. 276:251 (2001)); maleimides (e.g., Ro 31-8220, SB-216763, and SB-415286) (Coghlan et al., Chem. Biol. 7:793 (2000); Cross et al., J. Neurochem. 77:94 (2001); Hers et al., FEBS Lett. 460:433 (1999); Lochhead et al., Diabetes 50:937 (2001); Smith et al., Bioorg. Med. Chem. Lett. 11:635 (2001)); and muscarinic agonists (e.g., AF102B and AF150) (Forlenza et al., J. Neural. Transm. 107:1201 (2000)). Additional small molecule GSK-3 drug inhibitors compete with ATP, such as Aloisines (e.g., Aloisine A and Aloisine B) (Martinez, et al., J. Med. Chem. 45:1292 (2002); Martinez et al., Med. Res. Rev. 22:373 (2002); Mettey et al., J. Med. Chem. 46:222 (2003)). Small molecule inhibitors of GSK-3 also include CHIR 98014, CHIR 98021 and CHIR 99023 (Ring et al., Diabetes, 52:588 (2003); Nikoulina et al., Diabetes, 51:2190 (2002)).

Small molecule inhibitors of GSK-3 further include elements and ions such as lithium (Klein and Melton, Proc. Natl. Acad. Sci. USA 93:8455 (1996); and Stambolic et al., Curr. Biol. 6:1664 (1996)). Although fairly specific for GSK-3, a relatively high dose of lithium is required (Ki is mM) to inhibit GSK-3 activity in cell culture (Stambolic et al., Curr. Biol. 6:1664 (1996)). As with other elemental ions lithium acts by competition for Mg2+ (Ryves and Harwood Biochem. Biophys. Res. Commun. 280:720 (2001); Carmichael et al., J. Biol. Chem. 277:33791 (2002); and Stambolic et al., Curr. Biol. 6:1664 (1996)). The bivalent form of zinc, which mimics insulin action, also inhibits GSK-3 in cell culture at a concentration of 15 mM (Ilouz et al., Biochem. Biophys. Res. Commun. 295:102 (2002)). Another metal ion, beryllium, inhibits GSK-3 to half maximal activity at a concentration of 6 mM (Ryves et al., Biochem. Biophys. Res. Commun. 290:967 (2002)).

GSK-3 binding proteins are additional examples of GSK-3 inhibitors. For example, insulin inactivates GSK-3 through a phosphoinositide 3-kinase (PI 3-kinase)-dependent mechanism. PI-kinase-induced activation of PKB (also termed Akt) results in PKB phosphorylation of both GSK-3 isoforms (S9 of GSK-3b; S21 of GSK-3a) (Cross et al.,. Nature 378:785 (1995)), which inhibits GSK-3 activity. Other stimuli lead to inactivation of GSK-3 through S9/S21 phosphorylation, including growth factors such as EGF and PDGF that stimulate GSK-3-inactivating kinase p9ORSK (also known as MAPKAP-K1).

Further non-limiting examples of agents that reduce or inhibit expression or activity of a glycogenolytic enzyme include alpha-glucosidase inhibitors. Most of the known natural and synthetic alpha-glucosidase inhibitors are sugar analogs, such as pseudooligosaccharides (Bischoff, H., Eur. J. Clin. Investig. 24:3 (1994)), azasugars (Wong et al., J. Org. Chem. 60:1492 (1995)), and indolizidine alkaloids (Elbein, A. D., Ann. Rev. Biochem., 56:497 (1987)). Acarbose, a pseudotetrasaccharide from Actinoplanes species, is one of the most potent inhibitors of alpha-glucosidases (Legler G. Adv. Carb. Chem. Biochem., 48:319 (1990)). Its structure resembles the transition state of a substrate. As such, substrate analogues are a particular class of alpha-glucosidase inhibitors useful in accordance with the invention.

Additional non-limiting examples of agents that reduce or inhibit expression or activity of alpha-glucosidase include Bay ml 099 (Wisselaar et al., Clin. Chim. Acta., 182:41 (1989)), Conduritol B epoxide (Hermans et al., J. Biol. Chem. 266:13507 (1991)), Castanospermine (Rhinehart, et al., Biochem. Pharmacol. 41:223 (1991)), Isofagomine, a potent inhibitor of both the liver and muscle isoforms of glycogen phosphorylase (Dong et al., Biochem. 35:2788 (1996); Lundgren et al., Diabetes 45:S2 521 (1996); and Waagepetersen et al., Neurochemistry International 36:435 (2000)), Vilidamine, valienamine and valiolamine (Takeuchi et al., J. Biochem. 108:42 (1990); and U.S. Pat. No. 4,701,559), Acarviosine-glucose and isoacarbose (Kim et al., Arch. Biochem. Biophys. 371:277 (1999)), Salacinol, which can be isolated from a plant native to Sri Lanka (U.S. Pat. No. 6,455,573; and Yoshikawa et al., Bioorg. Med. Chem. 10:1547 (2002)), D(+)-trehalose (Matsuur et al., Biosci. Biotechnol. Biochem. 66:1576 (2002)), Callyspongynic acid (1) (Nakao et al.,J. Nat Prod. 65:922 (2002)), 1-Deoxynojirimcin (DNM) (Papandreou et al., Mol. Pharmacol. 61:186 (2002)), Touchi-extract (Hiroyuki et al., J. Nutr. Biochem. 12:351 (2001)), Diketopiperazine (1) (Kwon et al., J. Antibiot. 53:954 (2000); Sou et al., Chem. Pharm. Bull. 49:791 (2002)), 2,6-Dideoxy-7-O-(beta-D-glucopyranosyl)2,6-imino-D-glycero-L-gulo-heptitol(7-O-beta-D-glucopyranosyl-alpha-homonojirimycin, 1) (Ikeda et al., Carbohydr. Res. 323:73 (2000)), Ethanolamine and phenyl 6-deoxy-6-(morpholin-4-yl)-beta-D-glucopyranoside (Balbaa et al., Carbohydr. Res. 317:100 (1999)), N-methy-1-deoxynojirimycin (MOR-14) (Minatoguchi et al., Circulation, 97:1290 (1998)), Acavisonine-simmondsin (Baek et al., Biosci. Biotechnol. Biochem. 67:532 (2003)), Nestrisine (Tsujii et al., Biochem. Biophys. Res. Commun. 220:459 (1996)), Bay g 5421 (Aletor et al., Poult. Sci. 82:796 (2003)), Sangzhi (Ramulus mori, SZ),( Ye et al., Yao Xue Xue Bao, 37:108 (2002)), 2,4,6-trinitrophenyl 2-deoxy-2,2-difluoro-alpha-glucoside (Braun et al., J. Biol. Chem. 270:26778 (1995)), L-histidine, histamine and imidazole derivatives of (Field et al., Biochem. J. 274:885 (1991)), 4-O-alpha-D-glucopyranosylmoranoline and is various N-substituted derivatives (Yoshikuni et al., Chem. Pharm. Bull, 37:106 (1989)), Epicastanospermine (Molyneux et al., Arch. Biochem. Biophys., 251:450 (1986)), Nojirimycin (Chambers et al., Biochem. Biophys. Res. Commun. 107:1490 (1982)), and Nojirimycin tetrazole (Mitchell et al., Biochemistry, 35:7341 (1996)).

Further specific examples of alpha-glucosidase inhibitors include O-4,6-dideoxy-4-[[[1 S-(1 alpha,4alpha,5beta,6alpha)]-4,5,6-trihydroxy-3 (hydroxymethyl)-2-cyclohexen-1-yl]amino]-alpha-D-glucopyranosyl-(1-4)O-alpha-D-glucopyranosyl-(1-4)-D-glucose, also known as acarbose; 2(S),3(R), 4(S),5(S)-tetrahydroxy-N-[2-hydroxy-1-(hydroxymethyl)-ethyl]-5-(hydroxymethyl)-1(S)-cyclohexamine, also known as voglibose (A0-128) (Goke et al., Digestion, 56:493 (1995)); 1,5-dideoxy-1,5-[(2-hydroxyethyl)imino]-D-glucitol, also known as miglitol; 1,5-dideoxy-1,5-[2-(4-ethoxycarbonylphenoxy)ethylimino]-D-glucitol, also known as emiglitate (Lembcke et al., Res. Exp. Med. 191:389 (1991)); 2,6-dideoxy-2,6-imino-7-(beta-D-glucopyranosyl) -D-glycero-L-guloheptitol, also known as MDL-25637;1,5-dideoxy-1,5-(6-deoxy-1-O-methyl-alpha-D-glucopyranos-6-ylimino)-D-glucitol, also known as camiglibose; 1,5,9,11,14-pentahydroxy-3-methyl-8,13-dioxo-5,6,8,13-tetrahydrobenzo[a]naphthacene-2-carboxylic acid, also known pradimicin Q; adiposine; and 1,2-dideoxy-2-[2(S),3(S),4(R)-trihydroxy-5-(hydroxymethyl)-5-cyclohexen-1(S)-ylamino]-L-glucopyranose, also known as salbostatin. Indolizidine alkaloids, such as australine, castanospermine, and swainsonine are alpha-glucosidase inhibitors. Alpha-glucosidase inhibitors also include oral anti-diabetics (Lebovitz, H. E. Drugs, 44:21 (1992)). N-butyldeoxynojirimycin (N-butyl-DNJ) and related N-alkyl derivatives of DNJ are inhibitors of alpha-glucosidase I and II (Saunier et al., J. Biol. Chem. 257:14155 (1982); and Elbein, Ann. Rev. Biochem. 56:497 (1987)). 1,5-dideoxy-1,5-imino-D-glucitol and derivatives including N-alkyl, N-acyl, N-aroyl, N-aralkyl, and O-acyl derivatives are alpha-glucosidase inhibitors. Alpha-glucosidase inhibitors include L-arabinose and forms that are found in plants such as arabinan, arabinoxylan and arabinogalactan. Castanospermine is an example of an alpha-glucohydrolase inhibitor that is not readily reversible and has a relatively long duration of action. It also inhibits lysosomal alpha-glucosidase, which results in the accumulation of lysosomal glycogen. Another particular alpha-glucohydrolase inhibitor is 1,5-dideoxy-1,5->(6-deoxy-1-O-methyl-6-alpha,D-glucopyranosyl) imino-D-glucitol (MDL 73945) (Robinson et al, Diabetes 40:825 (1991)). Additional alpha-glucohydrolase inhibitors include glucopyranosyl and oligoglucosidyl derivatives of 4,6-bisdesoxy-4-(4,5,6-trihydroxy-3-hydroxymethylcyclohex-2-en-1-ylamino)-alpha-D-glucopyranose. The compound O-{4,6-bisdesoxy-4-[1S-(1,4,6/5)-4,5,6-trihydroxy-3-hydroxymethylcyclohex-2-en-1-ylamino]alpha-D-glucopyranosyl}-(1-4)-O-alpha-D-glucopyranosyl-( 1-4)-D-glucopyranose is a representative species (U.S. Pat. No. 4,062,950).

Table 1 below illustrates exemplary alpha-glucosidase inhibitors having structure of Formula I (see, U.S. Pat. Nos. 6,143,932 and 6,121,489). The subgroups of Formula I are as follows: R₁ and R₂ independently are a hydrogen atom, an amino protecting group, C₁ to C₁₂ acyl, C₃ to C₁₀ cycloalkyl, C₃ to C₆ heterocycle, C₁ to C₁₂ alkyl, C₁ to C₁₂ substituted alkyl, C₇ to C₁₆ alkylaryl, C₇ to C₁₆ substitued alkylaryl, a C₆ to C₁₅ alkyl heterocycle, or a substituted C₆ to C₁₅ alkyl heterocycle; R₃, R₅, and R₇ are independently a hydrogen atom, C₁ to C₁₂ alkyl, C₁ to C₁₂ substituted alkyl, phenyl, substituted phenyl, C₇ to C₁₆ alkylaryl, C₇ to C₁₆ substitued alkylaryl, a C₆ to C₁₅ alkyl heterocycle, or a substituted C₆ to C₁₅ alkyl heterocycle; R₄, R₆, and R₈ are independently a C₁ to C₁₈ substituent group; R₉ is a hydrogen atom; R₁₀ is optionally present as a C₁ to C₁₈ substituent group when R₁ and R₂ are other than a hydrogen atom or an amino protecting group; AA, BB, and CC are independently 0 to 5; and B is from 0 to 3. The stereochemistry at the carbons bonded to R₃, R₅, and R₇ are independently R or S or a mixture of the two; when B is 2 or 3, each R₄ and R₅ can be the same or different; when B is 0, each R₆ and R₈ is different; and either R₁ or R₂ can be taken with R₃; R₄ can be taken with R₅; R₆ can be taken with R₇; respectively and independently, to form a subtituted or unsubstituted pyrrolidine ring. X and Y are either each a hydrogen atom or taken together to represent a carbonyl group.

The IC50 values in Table 1 represent the concentration for 50% enzyme inhibition, and the assay was performed as previously described (Haslvorson and Ellias, Biochem. Biophys. Acta, 30:28 (1958)). The most active inhibitors are compounds of Formula I, wherein X and Y are taken together to form a carbonyl group, B is zero, AA, BB, and CC are zero except were noted, R₉ is a hydrogen atom, R₈ is benzyl, R₆ is naphth-2-ylmethyl, R₃ is S-(N-(naphth-2-ylmethyl)indol-3-ylmethyl), R_{1 and R}₂ are each hydrogen, R.₁₀ is absent, and R₇

TABLE 1

| alpha-Glucosidase | Inhibition |
|---|---|
| R₇ | IC50 (μM) |
| R-(4-(N-benzylamino)-n-butyl) | 17 |
| S-(4-(N-benzylamino)-n-butyl) | 19 |
| S-(3-guanidino)-n-propyl) | 38 |
| R-(3-guanidino)-n-propyl) | 38 |
| S-pyrrolidine (taken in | 141 |
| conjunction with R₈) | |
| S-methyl | 167 |
| Hydrogen atom | 167 |
| R-(2-methyl)propyl | 170 |
| S-(1-hydroxymethyl) | 176 |
| S-(phenyl) | 184 |
| S-(4-hydroxybenzyl) | 190 |
| R-methyl | 199 |
| S-benzyl | 328 |
| S-(2-methyl)propyl | 356 |
| S-(indol-3-ylmethyl) | 356 |
| S-(iso-propyl) | 356 |
| R-(2-methyl)prop-1-yl | 398 |
| S-4-hydroxyprrolidine (in | 437 |
| conjunction with R₈) | |
| S-(1-hydroxyethyl) | 460 |
| S-[N′,N′-dibenzylamido)ethyl]) | 529 |
| R-(4-hydroxybenzyl) | 540 |
| R-(iso-propyl) | 552 |
| R-(N′-benzyl indol-3-ylmethyl) | 552 |
| S-(2-(methylsulfinyl)ethyl) | 564 |
| S-(1-methyl)prop-1-yl | 610 |
| S-(N′-benzyl indol-3-ylmethyl) | 632 |
| S-(n-propyl) | 632 |
| R-(indol-3-ylmethyl) | 667 |
| S-(cyclohexylmethyl) | 667 |
| R′-(1-hydroxyethyl) | 678 |
| R-pyrrolidine (taken in | 702 |
| conjunction with R₈) | |
| S-[N′,N′-dibenzylamido)ethyl | 713 |
| R-(n-butyl) | 724 |
| hydrogen atom, AA = 1 | 770 |
| R-(n-propyl) | 828 |

Agents that increase intracellular glycogen levels additionally include, for example, Ochratoxin A (Dwivedi and Burns, Res. Vet. Sci. 36:92 (1984)), N-acetylcysteine (Itinose et al., Res. Commun. Chem. Pathol. Pharmacol. 83:87 (1994)), Dichloroacetate (DCA) (Kato-Weinstein et al., Toxicology, 130:141 (1998); Lingohr et al., Toxicol. Sci. 68:508 (2002)), Canthardin (Wang et al., Toxicology, 147:77 (2000)), Methylobromofenvinphos (IPO 63 compound) (Chishti and Rotkeiwicz, Arch. Environ. Contam. Toxicol. 22:445 (1992)), Genistein (Okazaki et al., (2002) Arch. Toxicol. 76:553), Quinine (al-Habori et al., Biochem. J. 282:789 (1992)), Alveld toxins (Flaoyen et al., Vet. Res. Commun. 15:443 (1991)), Methionine sulfoximine (Havor and Delorme Glia 4:64 (1991)), Tunicamycin (Chardin et al., Cell Tissue Res., 256:519 (1989)), Metformin (Detaille et al., Biochem. Pharmacol. 58:1475 (1999)), 5-idotubercidin (Fluckiger-Isler and Walter Biochem. J. 292:85 (1993)), Cantharidin (Wang et al., Toxicology, 147:77 (2000)), Diazoxide (Alemzadeh et al., Eur. J. Endocrinol. 146:871 (2002)).

Hormones are yet another example of agents that can increase intracellular glycogen levels. Specific non-limiting examples include epidermal growth factor (Bosch et al., Biochem. J. 239:523 (1986)), hydrocortisone (Black Am. J. Physiol. 254:G65 (1988)), noradrenaline, vasoactive intestinal peptide (Allaman et al., Glia, 30:382 (2000)), glucocorticoids (Laloux et al., Eur. J. Biochem. 136:175 (1983)) and insulin.

Dietary supplements are a further example of agents that can increase intracellular glycogen levels. Specific non-limiting examples include glucose (Watson et al., Biochemistry, 33:5745 (1994)), fructose (Gergely et al., Biochem. J., 232:133 (1985)), D-tagatose (Kruger et al., Regul. Toxicol. Pharmacol. 29: S1-S10 (1999)), oligofructose in combination with insulin (Flamm et al., Crit. Rev. Food Sci. Nutr. 41:353 (2001)), and Na+-co-transported amino acids such as glutamine, alanine, asparagine and proline (Hue L, Gaussin V. In: Amino Acid Metabolism and Therapy in Health and Nutritional Disease (Cynober, L. A., ed) pp. 179-188, CRC Press, Boca Raton, Fla. (1995)).

Plants and plant extracts are still another example of agents that can increase intracellular glycogen levels. Specific non-limiting examples include Rhamnus cathartica (Lichtensteiger et al., Toxicol. Pathol. 5:449 (1997)), Mormordica charantia and Mucuna (Rathi et al., Phytother Res. 16:236 (2002)), and powdered seed of Graninia Kola (Braide and Grill Gegenbaurs. Morphol. Jahrb. 136:95 (1990)).

In addition to the exemplary inhibitors disclosed herein and known in the art, glycogenolytic enzyme inhibitors can be designed based upon structure and function knowledge. For GSK-3, for example, the crystal structure has been determined (Bax et al., Structure (Camb) 9:1143 (2001); Dajani et al., Cell 105:721 (2001); ter Haar et al., Nat. Struct. Biol. 8:593 (2001)). Analysis of the GSK-3 crystal structure reveals that the enzyme prefers primed, pre-phosphorylated substrates. The T-loop of GSK-3 is tyrosine phosphorylated at Y216 and Y279 in GSK-3b and GSK-3a, respectively, but not threonine phosphorylated. Y216/Y279 phosphorylation may play a role in opening the substrate-binding site (Dajani et al., Cell 105:721 (2001)). Thus, T-loop tyrosine phosphorylation of GSK-3 may facilitate substrate phosphorylation but is not strictly required for kinase activity (Dajani et al., Cell 105:721 (2001)). The crystal structure of GSK-3 also indicates that the inhibitory role of S9/S21 serine phosphorylation is to create a primed pseudosubstrate that binds intramolecularly to the positively charged pocket. This folding precludes phosphorylation of substrates because the catalytic groove is occupied. The mechanism of inhibition is competitive and, therefore, pseudosubstrates in high enough concentrations can out-compete primed substrates and vice versa. Thus, small molecule inhibitors modeled to fit in the positively charged pocket of the GSK-3 kinase domain can selectively inhibit binding of primed substrates, such as glycogen synthase.

In addition to the crystal structure, studies indicate that GSK-3 has a preference for target proteins that are pre-phosphorylated at a ‘priming’ residue located C-terminal to the site of GSK-3 phosphorylation (Fiol et al., J. Biol. Chem. 262:14042 (1987)). The consensus sequence for GSK-3 substrates is Ser/Thr-X-X-XSer/Thr-P, where the first Ser or Thr is the target residue, X is any amino acid (but often Pro), and the last Ser-P/Thr-P is the site of priming phosphorylation. Priming phosphorylation increases the efficiency of substrate phosphorylation of most GSK-3 substrates by 100-1000-fold (Thomas et al., FEBS Lett. 458:247 (1999)). For example, glycogen synthase, the prototypical primed substrate, undergoes priming phosphorylation by casein kinase II (CK2) and then sequential multisite phosphorylation by GSK-3 (Fiol et al., Arch. Biochem. Biophys. 267:797 (1988); Fiol et al., J. Biol. Chem. 265:6061 (1990)). Some GSK-3 substrates lack a priming site. These proteins often display negatively charged residues at or near the priming position that may mimic a phospho-residue.

Because GSK-3 has many substrates, GSK-3 requires numerous levels of regulation to confer substrate specificity. Thus, GSK-3 can be inhibited via any of these signals. For example, GSK-3 can be inhibited through serine phosphorylation; inhibiting tyrosine phosphorylation or stimulating tyrosine dephosphorylation; indirect inhibition by covalent modification of substrates through priming phosphorylation; and inhibition or facilitation of GSK-3-mediated substrate phosphorylation through interaction of GSK-3 with binding or scaffolding proteins.

Alpha-glucosidase inhibitors can also be designed based upon structure and function knowledge. For example, the catalytic mechanism of alpha-glucosidase involves carbocation. Irreversible enzyme inhibition by compounds such as 2-deoxy-2-fluro-α-D-glucosylfluoride or 5-fluoro-α-D-glucosylfluoride are due to the inductive effect of fluoride at C-2 or C-5 of the glucose ring, which destabilizes the transition state glucosyl cation and promotes formation of a stable glucosyl-enzyme intermediate (Krasikov et al., Biochemistry, 66:267 (2001)). Alpha-glucosidase ligands imitating characteristic features of carbocation (negative charge and/or semi-chair conformation) act as inhibitors. δ-Gluconolacton possessing a semi-chair conformation is a competitive inhibitor of bovine liver alpha-glucosidase (Firsov LM, Biokhimiya, 43:2222 (1978)). Alpha-glucosidase inhibitors carrying a positive charge are more potent inhibitors. For example, Tris inhibits alpha-glucosidase activity (Krasikov et al., Biochemistry, 66:267 (2001)). Thus, any composition that imitates ligands characteristic of carbocation can be an agent that inhibits alpha-glucosidase, particularly those with a positive charge.

The six-member ring structure typical for indolizidine alkaloids (castonospermine, swainosonine) and also for deoxynojirimycin is not essential for glucosidase inhibition. Rather, the presence of nitrogen in the ring and the configuration of hydroxyl groups relative to nitrogen are the primary preconditions for inhibitory activity (Tropea et al., Biochemistry, 28:2027 (1989)). Manifestation of potent inhibition apparently requires hydrogen bonding between the imine nitrogen and a catalytic acid. For example, the transition of N₁-alkyl-D-glucosylamines to N₁-butyl-(or dodecyl)-D-gluconamidines is accompanied by ˜10-fold increase of the inhibitory effect; the inhibitor geometry changes from tetrahedral C₁-geometry to planar sp² amidine geometry. This is believed to be because protonated amidines cannot accept protons from the catalytic acid (Legler G, Finken M, Carbohydr. Res., 292:103 (1996)). The most active structures and, consequently, inhibitory agents, therefore will have nitrogen in the ring that maintain the configuration relative to the hydroxyl groups.

In the methods of the invention in which the amount of intracellular glycogen “increases,” this means that glycogen levels are greater within a given cell or plurality of cells. The term “accumulate,” when used in reference to glycogen, also refers to any increase in intracellular glycogen levels. When the terms are used in reference to a plurality of cells, not all cells may respond equivalently and accumulate glycogen. Thus, a portion of the cells may exhibit increased glycogen levels and a portion of the cells may not exhibit increased glycogen levels.

Increased intracellular glycogen levels may be transient or longer in duration, but typically will be of a sufficient amount to be toxic. Toxic levels of glycogen will result in reduced or decreased cell proliferation, growth, survival, or viability, or will produce one or more other characteristic features of glycogen toxicity. Characteristics of glycogen toxicity include, for example, morphological changes such as cell swelling due to glycogen condensation, increased numbers and size of lysosomes, structural changes in lysosomes characterized by a granular appearance, and nuclear accumulation of glycogen, to name a few. Toxic levels of glycogen can therefore be determined by assaying cell proliferation or growth rate (e.g., doubling time, cell cycle length, etc.), survival time (e.g., longevity), viability (lysis or apoptosis), or histological analysis.

Thus, the invention provides methods that increase glycogen to an amount that is toxic to the cell. In various aspects, toxicity is detected by inhibition or reduction of cell proliferation, growth or survival, or by assaying for a morphological change associated with glycogen toxicity, such as cell swelling, increased numbers of lysosomes, increased size of lysosomes, or a structural change in lysosomes.

Toxic levels of glycogen can also result in reduced cell viability. Thus, the invention provides methods that increase glycogen to an amount that causes lysis or apoptosis of the cell.

Intracellular levels of glycogen that are toxic will vary depending on the cell type because certain cell types, such as liver and muscle, tend to store greater amounts of glycogen. Consequently, in order to induce glycogen toxicity, absolute amounts of glycogen may be greater in cell types that normally have greater amounts of intracellular glycogen, such as in liver and muscle cells. For example, in asynchronous cultures of human colorectal adenocarcinoma cell lines (HT-29, HRT-18, SW-480, and Caco-2), the kinetics of glycogen accumulation were similar from one cell line to another, which was characterized by lower relative levels in the exponential phase of growth, followed by a 3- to 4-fold increase in stationary phase. In synchronized cultures of HT-29 and HRT-18 cell lines, both exhibited low glycogen quantities during S, G2, and M followed by an increase beginning with G1 and peaking (2.5 to 3 times the initial values) in the middle of G1. This was followed by a symmetrical decrease in the second half of G1. However, glycogen present in stationary and exponential phase was specific for each cell line: maximum values in Caco-2, HRT-18, HT-29, and SW-480 cells were, 258.5+/−6.9 (S.D.), 88.9+/−2.6, 87.5+/−3, and 17.5+/−1.8 microgram of glycogen per milligram of protein, respectively (Rousset et al., Cancer Res. 39 (2 Pt 1):531 (1979)). Glycogen levels can therefore vary based on cell type, with cells normally having greater absolute levels generally also requiring greater absolute levels of glycogen for toxicity.

Susceptibility to glycogen toxicity may also vary depending on the cell type. Thus, levels of glycogen even slightly above the normal range may be sufficient to induce toxicity in certain cell types, whereas in other cell types, a significant increase in glycogen level above the normal range may be needed in order to induce toxicity. In either case, glycogen toxicity can be determined using any of a variety of assays and morphological criteria disclosed herein or otherwise known in the art (see, e.g., Phillips et al., The Liver: An Atlas and Text of Ultrastructural Pathology. New York: Raven Press (1987); Lembcke et al., Res. Exp. Med. 191:389 (1991); and Baudhuin et al., Lab. Invest. 13:1139 (1964)).

In various embodiments of the invention, agents and treatments that have previously been characterized as stimulating or increasing activity of a glycogenic enzyme, inhibiting or decreasing activity of a glycogenolytic enzyme or modulating activity of a protein that directly or indirectly affects intracellular glycogen levels are applicable, provided that the agent or treatment is used in amounts that increase glycogen levels to toxic levels, including levels sufficient to kill target cells. That is, agents and treatments known in the art that have glycogenic enzyme stimulating activity, glycogenolytic enzyme inhibiting activity or that modulate activity of a protein that affects intracellular glycogen levels can be employed in accordance with the invention, when amounts of the agents and treatments used are sufficient to increase intracellular glycogen to toxic levels, or are sufficient to kill cells.

In additional embodiments of the invention, agents and treatments that are known to or that inherently stimulate or increase activity of a glycogenic enzyme, inhibit or decrease activity of a glycogenolytic enzyme, or modulate activity of another protein that in turn results in increased intracellular glycogen levels are applicable, provided that the agent or treatment has not been employed to treat a hyperproliferative cell or cell proliferative disorder (e.g., benign hyperplasia or a tumor or cancer) prior to the invention. That is, any agent or treatment known in the art and recognized to have, or that is known in the art and inherently has, the ability to stimulate or increase activity of a glycogenic enzyme, inhibit or decrease activity of a glycogenolytic enzyme or that modulates activity of a protein that results in increased intracellular glycogen levels can be employed in accordance with the invention, provided that the agents and treatments known in the art have not been used to treat a cell proliferative disorder prior to the invention. Optionally, such known agents and treatments used in accordance with the invention increase glycogen levels to toxic levels, including amounts sufficient to kill target cells.

The invention includes in vivo methods. For example, as described herein a cell such as a hyperoliferative cell can be present in a subject, such as a mammal (e.g., a human subject). The subject optionally has or is at risk of having a cell proliferative disorder. Hyperproliferative cells comprising the cell proliferativedisorder may be treated in accordance with the invention to increase intracellular glycogen thereby inducing toxicity.

As used herein, the terms “cell proliferative disorder,” “hyperproliferate,” “hyperproliferative disorder” and grammatical variations thereof, when used in reference to a cell, tissue or organ, refers to any undesirable, excessive or abnormal cell, tissue or organ proliferation, growth, differentiation or survival. A hyperproliferative cells denotes a cell whose proliferation, growth, or survival is greater than a corresponding reference normal cell, e.g., a cell of a cell proliferative disorder. Proliferative and differentiative disorders include diseases and physiological conditions, both benign hyperplastic conditions and neoplasia, characterized by undesirable, excessive or abnormal cell numbers, cell growth or cell survival in a subject. Specific examples of such disorders include metastatic and non-metastatic tumors and cancers.

Thus, the invention also provides methods of treating a cell proliferative disorder (e.g., benign hyperplasia or a tumor or cancer) in a subject. In one embodiment, a method of treating a cell proliferative disorder that is not a liver, muscle or brain cell disorder includes expressing in one or more cells comprising the disorder a gene product that increases the amount of intracellular glycogen, sufficient to treat the cell proliferative disorder. In another embodiment, a method of treating a cell proliferative disorder that is not a liver, muscle or brain cell disorder includes contacting one or more cells comprising the disorder with an agent that increases the amount of intracellular glycogen, sufficient to treat the cell proliferative disorder. In particular aspects, the cell proliferative disorder comprises a metastatic or non-metastatic cancer. In additional aspects, the cancer cell is present in head or neck, breast, esophagus, mouth, stomach, lung, gastrointestinal tract, pancreas, kidney, adrenal gland, bladder, colon, rectum, prostate, uterus, cervix, ovary, testes, skin, or hematopoetic system.

In yet another embodiment, a method of treating a cell proliferative disorder includes expressing in one or more cells comprising the disorder a gene product that increases the amount of intracellular glycogen, sufficient to treat the cell proliferative disorder. In still another embodiment, a method of treating a cell proliferative disorder includes contacting one or more cells comprising the disorder with an agent that increases the amount of intracellular glycogen, provided that the agent does not substantially inhibit activity or expression of a glycogen phosphorylase isotype, sufficient to treat the cell proliferative disorder. In particular aspects, the cell proliferative disorder comprises a metastatic or non-metastatic cancer. In additional aspects, the cancer cell is present in brain, head or neck, breast, esophagus, mouth, stomach, lung, gastrointestinal tract, liver, pancreas, kidney, adrenal gland, bladder, colon, rectum, prostate, uterus, cervix, ovary, testes, skin or muscle, or hematopoetic system.

Further provided are methods of treating a subject having or at risk of having a tumor. In one embodiment, the tumor is not a liver, muscle or brain tumor, and a method includes expressing in one or more of the tumor cells a gene product that increases the amount of intracellular glycogen, effective to treat the subject. In another embodiment, the tumor is not a liver, muscle or brain tumor, and a method includes contacting one or more of the tumor cells with an agent that increases the amount of intracellular glycogen, effective to treat the subject. In an additional embodiment, a method includes expressing in one or more of the tumor cells a gene product that increases the amount of intracellular glycogen, effective to treat the subject. In still another embodiment, a method includes contacting one or more of the tumor cells with an agent that increases the amount of intracellular glycogen, provided that the agent does not substantially inhibit activity or expression of a glycogen phosphorylase isotype, effective to treat the subject.

Additionally provided are methods of treating a subject undergoing or having undergone tumor therapy. In one embodiment, the tumor is not a liver, muscle or brain tumor, and a method includes administering to the subject an agent that increases the amount of intracellular glycogen in a cell, sufficient to treat the subject. In another embodiment, a method includes administering to the subject an agent that increases the amount of intracellular glycogen, provided that the agent does not substantially inhibit activity or expression of a glycogen phosphorylase isotype, sufficient to treat the subject.

As used herein, the terms “treat,” “treating,” “treatment” and grammatical variations thereof mean subjecting an individual patient to a protocol, regimen or process of the invention in which it is a desired to obtain a particular physiologic effect or outcome in that patient. Since every treated patient may not respond to a particular treatment protocol, treating does not require that the desired effect be achieved in any particular patient or patient population. In other words, a given patient or patient population may fail to respond to the treatment.

The terms “tumor,” “cancer,” and “neoplasia” are used interchangeably herein and refer to a cell or population of cells of any cell or tissue origin, whose growth, proliferation or survival is greater than growth, proliferation or survival of a normal counterpart cell. Such disorders include, for example, carcinoma, sarcoma, melanoma, neural (blastoma, glioma), and reticuloendothelial, lymphatic or haematopoietic neoplastic disorders (e.g., myeloma, lymphoma or leukemia). Tumors include both metastatic and non-metastatic types, and include any stage I, II, III, IV or V tumor, or a tumor that is in remission.

Tumors can arise from a multitude of primary tumor types, including but not limited to breast, lung, thyroid, head and neck, brain, adrenal gland, thyroid, lymph, gastrointestinal (mouth, esophagus, stomach, small intestine, colon, rectum), genito-urinary tract (uterus, ovary, cervix, bladder, testicle, penis, prostate), kidney, pancreas, liver, bone, muscle, skin, and may metastasize to secondary sites.

A “solid tumor” refers to neoplasia or metastasis that typically aggregates together and forms a mass. Specific examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. The term also includes carcinosarcomas, e.g., which include malignant tumors composed of carcinomatous and sarcomatous tissues. Adenocarcinoma includes a carcinoma of a glandular tissue, or in which the tumor forms a gland like structure. Melanoma refers to malignant tumors of melanocytes and other cells derived from pigment cell origin that may arise in the skin, the eye (including retina), or other regions of the body. Additional carcinomas can form from the uterine/cervix, lung, head/neck, colon, pancreas, testes, adrenal gland, kidney, esophagus, stomach, liver and ovary.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma

A “liquid tumor” refers to neoplasia of the reticuloendothelial or haematopoetic system, such as a lymphoma, myeloma, or leukemia, or a neoplasia that is diffuse in nature. Particular examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

Methods of the invention include methods providing a detectable or measurable improvement in a subject's condition: a therapeutic benefit. A therapeutic benefit is any objective or subjective, transient or temporary, or long term improvement in the condition, or a reduction in severity or adverse symptom of the disorder. Thus, a satisfactory clinical endpoint is achieved when there is an incremental or a partial reduction in the severity, duration or frequency of one or more associated adverse symptoms or complications, or inhibition or reversal of one or more of the physiological, biochemical or cellular manifestations or characteristics of the condition. A therapeutic benefit or improvement (“ameliorate” is used synonymously) therefore need not be complete destruction of all target proliferating cells (e.g., tumor) or ablation of all adverse symptoms or complications associated with a cell proliferative disorder. For example, partial destruction of a tumor cell mass, or even a stabilization of the tumor by inhibiting progression or worsening of the tumor, can reduce mortality and prolong lifespan even if only for a few days, weeks or months, even though a portion or the bulk of the tumor remains.

Specific non-limiting examples of therapeutic benefit include a reduction in tumor volume (size or cell mass), inhibiting an increase in tumor volume, slowing or inhibiting tumor progression or metastasis, stimulating, inducing or increasing tumor cell lysis or apoptosis. As disclosed herein, the effect of the invention methods may be to increase the tumor cell mass due to cell swelling induced by glycogen toxicity. A reduction in tumor cell mass may therefore occur after cell swelling subsides or cell lysis or apoptosis of the tumor occurs. Examination of a biopsied sample containing a tumor (e.g., blood or tissue sample), can establish whether tumor cells exhibit characteristic features of glycogen toxicity, or whether a reduction in numbers of tumor cells or inhibition of tumor cell proliferation, growth or survival has occurred. Alternatively, for a solid tumor, invasive and non-invasive imaging methods can ascertain tumor size or volume.

Additional adverse symptoms and complications associated with tumor, neoplasia, and cancer that can be reduced or decreased include, for example, nausea, lack of appetite, lethargy, pain and discomfort. Thus, a partial or complete reduction in the severity, duration or frequency of adverse symptoms, an improvement in the subjects subjective feeling, such as increased energy, appetite, psychological well being, are all specific non-limiting examples of therapeutic benefit

Treatments also considered effective are those that result in reduction of the use of another therapeutic regimen, protocol or process. For example, for a tumor, a method of the invention is considered as having a therapeutic benefit if its practice results in less frequent or reduced dose of an anti-tumor or immune enhancing therapy, such as a chemotherapeutic drug, radiotherapy, or immunotherapy, being required for tumor treatment.

Thus, in accordance with the invention, methods of increasing effectiveness of an anti-tumor therapy are provided. In one embodiment, a method includes administering to a subject that is undergoing or has undergone anti-tumor or immune-enhancing therapy not for a liver, muscle or brain tumor, an agent that increases the amount of intracellular glycogen, and an anti-tumor or immune-enhancing therapy. In another embodiment, a method includes administering to a subject that is undergoing or has undergone anti-tumor or immune-enhancing therapy, an agent that increases the amount of intracellular glycogen, provided that the agent does not substantially inhibit activity or expression of a glycogen phosphorylase isotype, and an anti-tumor or immune-enhancing therapy. The agent can be administered prior to, substantially contemporaneously with or following administration of and anti-tumor or immune-enhancing therapy.

The doses or an “amount effective” or “amount sufficient” for treatment to achieve a therapeutic benefit or improvement objectively or subjectively ameliorate one, several or all adverse symptoms or complications of the condition, to a measurable or detectable extent, although preventing or inhibiting a progression or worsening of the disorder, condition or adverse symptom, is a satisfactory outcome. Thus, in the case of a cell proliferative disorder, the amount will be sufficient to provide a therapeutic benefit to the subject or to ameliorate a symptom of the disorder. The dose may be proportionally increased or reduced as indicated by the status of the disorder being treated or any side effects of the treatment.

Of course, as is typical for any treatment protocol, subjects will exhibit a range of responses to treatment. Appropriate amounts will therefore depend at least in part upon the disorder treated (e.g., benign hyperplasia or a tumor, and the tumor type or stage), the therapeutic effect desired, as well as the individual subject (e.g., the bioavailability within the subject, gender, age, etc.) and the subject's response to the drug based upon genetic and epigenetic variability (e.g., pharmacogenomics).

The terms “subject” and “patient” are used interchangeably herein and refer to animals, typically mammals, such as a non-human primates (gorilla, chimpanzee, orangutan, macaque, gibbon), domestic animals (dog and cat), farm and ranch animals (horse, cow, goat, sheep, pig), laboratory and experimental animals (mouse, rat, rabbit, guinea pig) and humans. Subjects include disease model animals (e.g., such as mice and non-human primates) for studying in vivo efficacy (e.g., a tumor or cancer animal model). Human subjects include adults, and children, for example, newborns and older children, between the ages of 1 and 5, 5 and 10 and 10 and 18, and the elderly, for example, between the ages of 60 and 65, 65 and 70 and 70 and 100.

Subjects include humans having or at risk of having a cell proliferative disorder. Subjects also include candidates for an anti-tumor or immune enhancing therapy, subjects undergoing an anti-tumor or immune enhancing therapy, and subjects having undergone an anti-tumor or immune enhancing therapy.

At risk subjects include those with a family history, genetic predisposition towards, or have suffered a previous affliction with a cell proliferative disorder (e.g., a benign hyperplasia, tumor or cancer). At risk subjects further include environmental exposure to carcinogens or mutagens, such as smokers, or those in an industrial or work setting. Such subjects have either not been diagnosed or have not exhibited symptoms of the cell proliferative disorder. Thus, subjects at risk for developing a cell proliferative disorder such as cancer can be identified with genetic screens for tumor associated genes, gene deletions or gene mutations. Subjects at risk for developing breast cancer lack Brca1, for example. Subjects at risk for developing colon cancer have deleted or mutated tumor suppressor genes, such as adenomatous polyposis coli (APC′), for example. At risk subjects having particular genetic predisposition towards cell proliferative disorders are known in the art (see, e.g., The Genetic Basis of Human Cancer 2^{nd} ed. by Bert Vogelstein (Editor), Kenneth W. Kinzler (Editor) (2002) McGraw-Hill Professional; The Molecular Basis of Human Cancer. Edited by W B Coleman and G J Tsongalis (2001) Humana Press; and The Molecular Basis of Cancer. Mendelsohn et al., W B Saunders (1995)).

At risk subjects can therefore be treated prophylactically in order to inhibit or reduce the likelihood of developing a cell proliferative disorder, or after having been cured of a cell proliferative disorder, suffering a relapse of the same or a different cell proliferative disorder. The result of such treatment can be partial or complete prevention of a cell proliferative disorder, or an adverse symptom thereof in the treated at risk subject.

Nucleic acids useful in the invention include sequences encoding any protein that increases synthesis or intracellular amounts of glycogen, or that directly or indirectly contributes to glycogen accumulation. Such sequences therefore include sequences encoding any and all glycogenic enzymes and inhibitory nucleic acids of any and all glycogenolytic enzymes, as set forth herein.

Additional nucleic acid sequences useful in the invention include sequences encoding proteins that directly or indirectly modulate expression or activity of any protein that participates in intracellular glycogen accumulation. Particular examples include proteins that increase expression or activity of a glycogenic enzyme, and proteins that reduce expression or activity of a glycogenolytic enzyme. Such sequences therefore include proteins that regulate transcription or translation of glycogenic and glycogenolytic enzymes. One specific example of such a protein is Notch-1/Hes-1, which represses glycogenolytic enzyme α-glucosisdase gene expression (Yan et al., J Biol Chem., 277:29760 (2002)). Accordingly, nucleic acids encoding such proteins or targeting such proteins for inhibition can also be used in accordance with the invention.

The terms “nucleic acid,” “polynucleotide” refers to at least two or more ribo- or deoxy-ribonucleic acid base pairs (nucleotides) that are linked through a phosphoester bond or equivalent. Nucleic acids include polynucleotides and polynucleosides. Nucleic acids include single, double or triplex, circular or linear, molecules. A nucleic acid molecule may belong exclusively or in a mixture to any group of nucleotide-containing molecules, as exemplified by, but not limited to: RNA, DNA, cDNA, genomic nucleic acid, non-genomic nucleic acid, naturally occurring and non naturally occurring nucleic acid and synthetic nucleic acid.

Nucleic acids can be of any length. Nucleic acid lengths useful in the invention typically range from about 20 nucleotides to 20 Kb, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length. Shorter polynucleotides are commonly referred to as “oligonucleotides” or “probes” of single- or double-stranded DNA. However, there is no upper limit to the length of such oligonucleotides.

Polynucleotides include L- or D-forms and mixtures thereof, which additionally may be modified to be resistant to degradation when administered to a subject. Particular examples include 5′ and 3′ linkages that are resistant to endonucleases and exonucleases present in various tissues or fluids of a subject.

Nucleic acids include antisense. As used herein, the term “antisense” refers to a polynucleotide or peptide nucleic acid capable of binding to a specific DNA or RNA sequence. Antisense includes single, double, triple or greater stranded RNA and DNA polynucleotides and peptide nucleic acids (PNAs) that bind RNA transcript or DNA. Particular examples include RNA and DNA antisense that binds to sense RNA. For example, a single stranded nucleic acid can target a protein transcript that participates in metabolism, catabolism, removal or degradation of glycogen from a cell (e.g., mRNA). Antisense molecules are typically 100% complementary to the sense strand but can be “partially” complementary, in which only some of the nucleotides bind to the sense molecule (less than 100% complementary, e.g., 95%, 90%, 80%, 70% and sometimes less).

Triplex forming antisense can bind to double strand DNA thereby inhibiting transcription of the gene. Oligonucleotides derived from the transcription initiation site of the gene, e.g., between positions −10 and +10 from the start site, are a particular example.

Short interfering RNA (referred to as siRNA or RNAi) for inhibiting gene expression is known in the art (see, e.g., Kennerdell et al., Cell 95:1017 (1998); Fire et al., Nature, 391:806 (1998); WO 02/44321; WO 01/68836; WO 00/44895, WO 99/32619, WO 01/75164, WO 01/92513, WO 01/29058, WO 01/89304, WO 02/16620; and WO 02/29858). RNAi silencing can be induced by a nucleic acid encoding an RNA that forms a “hairpin” structure or by expressing RNA from each end of an encoding nucleic acid, making two RNA molecules that hybridize.

Ribozymes, which are enzymatic RNA molecules that catalyze the specific cleavage of RNA can be used to inhibit expression of the encoded protein. Ribozymes form sequence-specific hybrids with complementary target RNA, which is then cleaved. Specific examples include engineered hammerhead motif ribozyme molecules that can specifically and efficiently catalyze endonucleolytic cleavage of sequences encoding a protein that participates in metabolism, catabolism, removal or degradation of glycogen, for example.

Ribozyme cleavage sites within a potential RNA target can be initially identified by scanning the target molecule for cleavage sites which include, for example, GUA, GUU, and GUC. Once identified, RNA sequences of between about 15 and 20 ribonucleotides corresponding to the region of the target containing the cleavage site are evaluated for secondary structural features which may render the oligonucleotide inoperable. The suitability of candidate target sequences may also be evaluated by testing accessibility to hybridization with complementary oligonucleotides using ribonuclease protection assays.

Antisense, ribozymes, RNAi and triplex forming nucleic acid are referred to collectively herein as “inhibitory nucleic acid” or “inhibitory polynucleotides.” Such inhibitory nucleic acid can inhibit expression of a protein that participates in metabolism, catabolism, removal or degradation of intracellular glycogen, such as a glycogenolytic enzyme. Such inhibitory nucleic acid can inhibit expression or activity of a protein that in turn inhibits expression or activity of a protein that contributes to synthesis or accumulation of glycogen. By inhibiting expression or activity of such a protein, repression of the protein that participates in synthesis or accumulation of glycogen is relieved and intracellular glycogen accumulates.

Inhibitory polynucleotides do not require expression control elements to function in vivo. Such molecules can be absorbed by the cell or enter the cell via passive diffusion. Such molecules may also be introduced into a cell using a vector, such as a virus vector. Inhibitory polynucleotides may be encoded by a nucleic acid so that it is transcribed. Furthermore, such a nucleic acid encoding an inhibitory polynucleotide may be operatively linked to an expression control element for sustained or increased expression of the encoded antisense in cells or in vivo.

Inhibitory nucleic acid can be designed based on gene sequences available in the database. For example, as set forth herein, Genbank sequences for exemplary glycogenolytic enzymes are known in the art and can be used to design inhibitory nucleic acid.

Specific inhibitory nucleic acids are also known in the art. Particular examples of antisense for glycogenolytic enzymes include phosphorylase kinase alpha 2 expression modulation (U.S. Pat. No. 6,458,591); phosphorylase kinase alpha 1 expression modulation (U.S. Pat. No. 6,426,188); inhibition of phosphorylase kinase beta expression (U.S. Pat. No. 6,368,856); glycogen synthase kinase 3 beta expression modulation (U.S. Pat. No. 6,323,029); inhibition of glycogen synthase kinase 3 alpha expression (U.S. Pat. No. 6,316,259); and modulation of liver glycogen phosphorylase expression (U.S. Pat. No. 6,043,091).

Particular examples of siRNA inhibition include GSK3alpha and GSK3beta (Yu et al., Mol Ther. 7:228 (2003)). Inhibition of either GSK-3alpha or GSK-3beta by transfection of hairpin siRNA vectors produced elevated expression of the GSK-3 target beta-catenin, and inhibition of both kinases led to more pronounced beta-catenin expression, indicating vector-based siRNA inhibition of GSK-3alpha and GSK-3beta.

Nucleic acids further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode amino acid sequences of glycogenic enzymes. Other examples are nucleic acids complementary to a sequence that encodes an amino acid sequence of a glycogenic enzyme.

Nucleic acid deletions (subsequences and fragments) can have from about 10 to 25, 25 to 50 or 50 to 100 nucleotides. Such nucleic acids are useful for expressing polypeptide subsequences, for genetic manipulation (as primers and templates for PCR amplification), and as probes to detect the presence or an amount of a sequence encoding a protein (e.g., via hybridization), in a cell, culture medium, biological sample (e.g., tissue, organ, blood or serum), or in a subject.

The term “hybridize” and grammatical variations thereof refers to the binding between nucleic acid sequences. Hybridizing sequences will generally have more than about 50% homology to a nucleic acid that encodes an amino acid sequence of a reference sequence. The hybridization region between hybridizing sequences can extend over at least about 10-15 nucleotides, 15-20 nucleotides, 20-30 nucleotides, 30-50 nucleotides, 50-100 nucleotides, or about 100 to 200 nucleotides or more.

Nucleic acids can be produced using various standard cloning and chemical synthesis techniques. Such techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., microorganism, such as yeast or bacteria, a eukaryote such as an animal or mammalian cell or in a plant).

For expression or manipulation, nucleic acids can be incorporated into expression cassettes and vectors. Expression cassettes and vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

Physical linkage is not required for the elements to be operably linked. For example, a minimal element can be linked to a nucleic acid encoding a glycogenic enzyme. A second element that controls expression of an operably linked nucleic acid encoding a protein that functions “in trans” to bind to the minimal element can influence expression of the glycogenic enzyme. Because the second element regulates expression of the glycogenic enzyme, the second element is operably linked to the nucleic acid encoding the glycogenic enzyme even though it is not physically linked.

The term “expression control element” refers to nucleic acid that influences expression of an operably linked nucleic acid. Promoters and enhancers are particular non-limiting examples of expression control elements. A “promotor sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of MRNA, polyadenylation signal to provide proper polyadenylation of the transcript of interest, and stop codons.

Expression control elements include “constitutive” elements in which transcription of an operably linked nucleic acid occurs without the presence of a signal or stimuli. Expression control elements that confer expression in response to a signal or stimuli, which either increases or decreases expression of the operably linked nucleic acid, are “regulatable.” A regulatable element that increases expression of the operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically active in specific cell or tissue types because they are recognized by transcriptional activator proteins, or other regulators of transcription, that are active in the specific cell or tissue type as compared to other cell or tissue types.

Tissue-specific expression control elements include promoters and enhancers active in hyperproliferative cells, such as cell proliferative disorders including tumors and cancers. Particular non-limiting examples of such promoters are hexokinase II, COX-2, alpha-fetoprotein, carcinoembryonic antigen, DE3/MUC1, prostate specific antigen, C-erB2/neu, telomerase reverse transcriptase and hypoxia-responsive promoter.

For bacterial expression, constitutive promoters include T7, as well as inducible promoters such as pL of bacteriophage λ, plac, ptrp, ptac (ptrp-lac hybrid promoter). In insect cell systems, constitutive or inducible promoters (e.g., ecdysone) may be used. In yeast, constitutive promoters include, for example, ADH or LEU2 and inducible promoters such as GAL (see, e.g., Ausubel et al., In: Current Protocols in Molecular Biology, Vol. 2, Ch. 13, ed., Greene Publish. Assoc. & Wiley Interscience, 1988; Grant et al., In: Methods in Enzymology, 153:516-544 (1987), eds. Wu & Grossman, 1987, Acad. Press, N.Y.; Glover, DNA Cloning, Vol. II, Ch. 3, IRL Press, Wash., D.C., 1986; Bitter, In: Methods in Enzymology, 152:673-684 (1987), eds. Berger & Kimmel, Acad. Press, N.Y.; and, Strathern et al., The Molecular Biology of the Yeast Saccharomyces eds. Cold Spring Harbor Press, Vols. I and II (1982)).

For mammalian expression, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; the inducible mouse mammary tumor virus LTR) are used.

The invention methods, inter alia, therefore include introducing nucleic acid or protein into target cells, e.g., cells of a cell proliferative disorder. Such cells are referred to as transformed cells. The term “transformed,” when use in reference to a cell or organism, means a genetic change in a cell following incorporation of an exogenous molecule, for example, a protein or nucleic acid (e.g., a transgene) into the cell. Thus, a “transformed cell” is a cell into which, or a progeny of which an exogenous molecule has been introduced by the hand of man, for example, by recombinant DNA techniques. The nucleic acid or protein can be stably or transiently expressed in the transformed cell and progeny thereof. The transformed cell(s) can be propagated and the introduced protein expressed, or nucleic acid transcribed or encoded protein expressed. A progeny cell may not be identical to the parent cell, since there may be mutations that occur during replication.

Transformed cells include but are not limited to prokaryotic and eukaryotic cells such as bacteria, fungi, plant, insect, and animal (e.g., mammalian, including human) cells. In one particular aspect, the cell is a cell that can produce glycogen or is susceptible to glycogen toxicity. In another particular aspect, the cell is a cell that includes an expression control element of a glycogenic enzyme, glycogenolytic enzyme or other protein that participates in increasing or decreasing intracellular glycogen, operably linked to a reporter. The cells may be present in culture, part of a plurality of cells, or a tissue or organ ex vivo or in a subject (in vivo).

Typically, cell transformation employs a “vector,” which refers to a plasmid, virus, such as a viral vector, or other vehicle known in the art that can be manipulated by insertion or incorporation of a nucleic acid. For genetic manipulation “cloning vectors” can be employed, and to transcribe or translate the inserted polynucleotide “expression vectors” can be employed. Such vectors are useful for introducing nucleic acids, including nucleic acids that encode a glycogenic enzyme and nucleic acids that encode inhibitory nucleic acid, operably linked to an expression control element, and expressing the encoded protein or inhibitory nucleic acid (e.g., in solution or in solid phase), in cells or in a subject in vivo.

A vector generally contains an origin of replication for propagation in a cell. Control elements, including expression control elements as set forth herein, present within a vector, can be included to facilitate transcription and translation, as appropriate.

Vectors can include a selection marker. A “selection marker” is a gene that allows for the selection of cells containing the gene. “Positive selection” refers to a process in which cells that contain the selection marker survive upon exposure to the positive selection. Drug resistance is one example of a positive selection marker; cells containing the marker will survive in culture medium containing the selection drug, and cells lacking the marker will die. Selection markers include drug resistance genes such as neo, which confers resistance to G418; hygr, which confers resistance to hygromycin; and puro which confers resistance to puromycin. Other positive selection marker genes include genes that allow identification or screening of cells containing the marker. These genes include genes for fluorescent proteins (GFP and GFP-like chromophores, luciferase), the lacZ gene, the alkaline phosphatase gene, and surface markers such as CD8, among others. “Negative selection” refers to a process in which cells containing a negative selection marker are killed upon exposure to an appropriate negative selection agent. For example, cells which contain the herpes simplex virus-thymidine kinase (HSV-tk) gene (Wigler et al., Cell 11:223 (1977)) are sensitive to the drug gancyclovir (GANC). Similarly, the gpt gene renders cells sensitive to 6-thioxanthine.

Viral vectors included are those based on retroviral, adeno-associated virus (AAV), adenovirus, reovirus, lentivirus, rotavirus genomes, simian virus 40 (SV40) or bovine papilloma virus (Cone et al., Proc. Natl. Acad. Sci. USA 81:6349 (1984); Eukarvotic Viral Vectors, Cold Spring Harbor Laboratory, Gluzman ed., 1982; Sarver et al., Mol. Cell. Biol. 1:486 (1981)). Adenovirus efficiently infects slowly replicating and/or terminally differentiated cells and can be used to target slowly replicating and/or terminally differentiated cells. Additional viral vectors useful for expression include parvovirus, Norwalk virus, coronaviruses, paramyxo- and rhabdoviruses, togavirus (e.g., sindbis virus and semliki forest virus) and vesicular stomatitis virus (VSV).

Mammalian expression vectors include those designed for in vivo and ex vivo expression, such as AAV (U.S. Pat. No. 5,604,090). AAV vectors have previously been shown to provide expression in humans at levels sufficient for therapeutic benefit (Kay et al., Nat. Genet. 24:257 (2000); Nakai et al., Blood 91:4600 (1998)). Adenoviral vectors (U.S. Pat. Nos. 5,700,470, 5,731,172 and 5,928,944), herpes simplex virus vectors (U.S. Pat. No. 5,501,979) retroviral (e.g., lentivirus vectors are useful for infecting dividing as well as non-dividing cells and foamy viruses) vectors (U.S. Pat. Nos. 5,624,820, 5,693,508, 5,665,577, 6,013,516 and 5,674,703 and WIPO publications WO92/05266 and WO92/14829) and papilloma virus vectors (e.g., human and bovine papilloma virus) have all been employed in gene therapy (U.S. Pat. No. 5,719,054). Vectors also include cytomegalovirus (CMV) based vectors (U.S. Pat. No. 5,561,063). Vectors that efficiently deliver genes to cells of the intestinal tract have been developed (U.S. Pat. Nos. 5,821,235, 5,786,340 and 6,110,456).

A viral particle or vesicle containing the viral or mammalian vector can be designed to be targeted to particular cell types (e.g., undesirably proliferating cells) by inclusion of a protein on the surface that binds to a target cell ligand or receptor. Alternatively, a cell type-specific promoters and/or enhancer can be included in the vector in order to express the nucleic acid in target cells. Thus, the viral vector itself, or a protein on the viral surface can be made to target cells for transformation in vitro, ex vivo or in vivo.

Introduction of compositions (e.g., proteins and nucleic acids) into target cells can also be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (see, e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127).

Polymeric substances, microcapsules and colloidal dispersion systems such as liposomes are collectively referred to herein as “vesicles.” Accordingly, viral and non-viral vector means of delivery into cells or tissue, in vitro, in vivo and ex vivo are included.

The terms “protein,” “polypeptide” and “peptide” are used interchangeably herein to refer to two or more covalently linked amino acids, or “residues,” through an amide bond or equivalent. Polypeptides are of unlimited length and the amino acids may be linked by non-natural and non-amide chemical bonds including, for example, those formed with glutaraldehyde, N-hydoxysuccinimide esters, bifunctional maleimides, or N,N′-dicyclohexylcarbodiimide (DCC). Non-amide bonds include, for example, ketomethylene, aminomethylene, olefin, ether, thioether and the like (see, e.g., Spatola in Chemistry and Biochemistry of Amino Acids, Peptides and Proteins, Vol. 7, pp 267-357 (1983), “Peptide and Backbone Modifications,” Marcel Decker, N.Y.).

The term “isolated,” when used as a modifier of a composition, means that the compositions are made by the hand of man or are separated from their naturally occurring in vivo environment. Generally, compositions so separated are substantially free of one or more materials with which they normally associate with in nature, for example, one or more protein, nucleic acid, lipid, carbohydrate, cell membrane. The term “isolated” does not exclude alternative physical forms, such as polypeptide multimers, post-translational modifications (e.g., phosphorylation, glycosylation) or derivatized forms.

An “isolated” composition can also be “substantially pure” when free of most or all of the materials with which it typically associates with in nature. Thus, an isolated molecule that also is substantially pure does not include polypeptides or polynucleotides present among millions of other sequences, such as antibodies of an antibody library or nucleic acids in a genomic or cDNA library, for example. A “substantially pure” molecule can be combined with one or more other molecules. Thus, the term “substantially pure” does not exclude combinations of compositions.

Substantial purity can be at least about 60% or more of the molecule by mass. Purity can also be about 70% or 80% or more, and can be greater, for example, 90% or more. Purity can be determined by any appropriate method, including, for example, UV spectroscopy, chromatography (e.g., HPLC, gas phase), gel electrophoresis (e.g., silver or coomassie staining) and sequence analysis (nucleic acid and peptide).

Nucleic acids, proteins, agents and other compositions useful in accordance with the invention include modified forms as set forth herein, provided that the modified form retains, at least a part of, a function or activity of the unmodified or reference nucleic acid, protein, agent or composition. For example, a nucleic acid encoding a modified protein that participates in glycogen synthesis (e.g., a glycogenic enzyme) can retain sufficient activity to stimulate or increase intracellular glycogen (the modified protein can be used alone or in combination with another protein that participates in glycogen synthesis), but have increased or decreased activity relative to a reference unmodified protein that participates in glycogen synthesis.

Thus, the invention further employs proteins, nucleic acids, agents and other compositions having modifications of the exemplary proteins, nucleic acids, agents and compositions. As used herein, the term “modify” and grammatical variations thereof, when used in reference to a composition such as a protein, nucleic acid, agent, or other composition means that the modified composition deviates from a reference composition. Such modified proteins, nucleic acids, agents and other compositions may have greater or less activity than a reference unmodified protein, nucleic acid, agent or composition.

Polypeptide modifications include amino acid substitutions, additions and deletions, which are also referred to as “variants.” Polypeptide modifications also include one or more D-amino acids substituted for L-amino acids (and mixtures thereof), structural and functional analogues, for example, peptidomimetics having synthetic or non-natural amino acids or amino acid analogues and derivatized forms.

Polypeptide modifications further include fusion (chimeric) polypeptide sequences, which is an amino acid sequence having one or more molecules not normally present in a reference native (wild type) sequence covalently attached to the sequence, for example, one or more amino acids. Modifications include cyclic structures such as an end-to-end amide bond between the amino and carboxy- terminus of the molecule or intra- or inter-molecular disulfide bond. Polypeptides including antibodies may be modified in vitro or in vivo, e.g., post-translationally modified to include, for example, sugar residues, phosphate groups, ubiquitin, fatty acids or lipids.

A “conservative substitution” is the replacement of one amino acid by a biologically, chemically or structurally similar residue. Biologically similar means that the substitution is compatible with biological activity, e.g., enzyme activity. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

The term “identical” or “identity” means that two or more referenced entities are the same. Thus, where two protein sequences are identical, they have the same amino acid sequence. An “area of identity” refers to a portion of two or more referenced entities that are the same. Thus, where two protein sequences are identical over one or more sequence regions they share amino acid identity in that region. The term “substantial identity” means that the molecules are structurally identical or have at least partial function of one or more of the functions (e.g., a biological function) of the reference molecule. Polypeptides having substantial identity include amino acid sequences with 50%, 60%, 70%, 75%, 80%, 85%, 90%, 95%, 96%, 97%, 98%, or more identity to a reference polypeptide, provided that modified polypeptide has at least partial activity, e.g., contributes to glycogen synthesis or accumulation.

As used herein, the term “subsequence” or “fragment” means a portion of the full length molecule. A protein subsequence has one or more fewer amino acids than a full length comparison sequence (e.g. one or more internal or terminal amino acid deletions from either amino or carboxy-termini). A nucleic acid subsequence has at least one less nucleotide than a full length comparison nucleic acid sequence. Subsequences therefore can be any length up to the full length molecule.

Modified forms further include derivatized sequences, for example, amino acids in which free amino groups form amine hydrochlorides, p-toluene sulfonyl groups, carbobenzoxy groups; the free carboxy groups from salts, methyl and ethyl esters; free hydroxl groups that form O-acyl or O-alkyl derivatives, as well as naturally occurring amino acid derivatives, for example, 4-hydroxyproline, for proline, 5-hydroxylysine for lysine, homoserine for serine, omithine for lysine, etc. Modifications can be produced using any of a variety of methods well known in the art (e.g., PCR based site-directed, deletion and insertion mutagenesis, chemical modification and mutagenesis, cross-linking, etc.).

Polypeptide sequences can be made using recombinant DNA technology of polypeptide encoding nucleic acids via cell expression or in vitro translation, or chemical synthesis of polypeptide chains using methods known in the art. Polypeptide sequences can also be produced by a chemical synthesizer (see, e.g., Applied Biosystems, Foster City, Calif.).

The invention can be practiced in association with any other therapeutic regimen or treatment protocol. The invention compositions and methods also can be combined with any other agent or treatment that provides a desired effect. Exemplary agents and treatments have anti-tumor activity or immune enhancing activity.

The invention therefore provides methods in which the methods of the invention are used in combination with any therapeutic regimen or treatment protocol, such as an anti-cell proliferative protocol set forth herein or known in the art. In one embodiment, a method includes administering an anti-tumor or immune enhancing treatment or agent. The anti-tumor or immune enhancing treatment or agent can be administered prior to, substantially contemporaneously with or following administration of a nucleic acid or agent or treatment that increases intracellular glycogen.

As used herein, an “anti-tumor,” “anti-cancer” or “anti-neoplastic” agent, treatment, therapy, activity or effect means any agent, therapy, treatment regimen, protocol or process that inhibits, decreases, slows, reduces or prevents hyperplastic, tumor, cancer or neoplastic growth, metastasis, proliferation or survival. Anti-tumor agents, therapies or treatments can operate by disrupting, inhibiting or delaying cell cycle progression or cell proliferation; stimulating or enhancing apoptosis, lysis or cell death; inhibiting nucleic acid or protein synthesis or metabolism; inhibiting cell division; or decreasing, reducing or inhibiting cell survival, or production or utilization of a cell survival factor, growth factor or signaling pathway (extracellular or intracellular).

Examples of anti-tumor therapy include chemotherapy, immunotherapy, radiotherapy (ionizing or chemical), local or regional thermal (hyperthermia) therapy and surgical resection.

Specific non-limiting classes of anti-cell proliferative and anti-tumor agents include alkylating agents, anti-metabolites, plant extracts, plant alkaloids, nitrosoureas, hormones, nucleoside and nucleotide analogues. Specific non-limiting examples of microbial toxins include bacterial cholera toxin, pertussis toxin, anthrax toxin, diphtheria toxin, and plant toxin ricin. Specific examples of drugs include cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin and dibromomannitol.

Radiotherapy includes internal or external delivery to a subject. For example, alpha, beta, gamma and X-rays can administered to the subject externally without the subject internalizing or otherwise physically contacting the radioisotope. Specific examples of X-ray dosages range from daily doses of 50 to 200 roentgens for prolonged periods of time (3 to 5/week), to single doses of 2000 to 6000 roentgens. Dosages vary widely, and depend on duration of exposure, the half-life of the isotope, the type of radiation emitted, the cell type and location treated and the progressive stage of the disease. Specific non-limiting examples of radionuclides include, for example, ⁴⁷Sc ⁶⁷CU, ⁷²Se, ⁸⁸Y, ⁹⁰Sr, ⁹⁰Y, ⁹⁷Ru, ⁹⁹Tc, ¹⁰⁵Rh, ¹¹¹In, ¹²⁵I, ¹³¹I, ¹⁴⁹Tb, ¹⁵³Sm, ¹⁸⁶Re, ¹⁸⁸Re, ¹⁹⁴Os, ²⁰³Pb, ²¹¹At ²¹²Bi ²¹³Bi ²¹²Pb, ²²³Ra,²²⁵Ac ²²⁷Ac and ²²⁸Th.

As used here, the term “immune enhancing,” when used in reference to an agent, therapy or treatment, means that the agent, therapy or treatment, provides an increase, stimulation, induction or promotion of an immune response, humoral or cell-mediated. Such therapies can enhance immune response generally, or enhance immune response to a specific target, e.g., a cell proliferative disorder such as a tumor or cancer.

Specific non-limiting examples of immune enhancing agents include growth factors, survival factors, differentiative factors, cytokines and chemokines. An additional example is monoclonal, polyclonal antibody and mixtures thereof. Antibodies that bind to tumor cells via a tumor-associated antigen (TAA) are a particular example of an immune-enhancing treatment. The term “tumor associated antigen” or “TAA” refers to an antigen expressed by a tumor cell.

Particular examples of TAAs that can be targeted and corresponding antibodies include, for example, M195 antibody which binds to leukemia cell CD33 antigen (U.S. Pat. No. 6,599,505); monoclonal antibody DS6 which binds to ovarian carcinoma CA6 tumor-associated antigen (U.S. Pat. No. 6,596,503); human IBD12 monoclonal antibody which binds to epithelial cell surface H antigen (U.S. Pat. No. 4,814,275); and BR₉₆ antibody which binds to Le^{x} carbohydrate epitope expressed by colon, breast, ovary, and lung carcinomas. Additional anti-tumor antibodies that can be employed include, for example, Herceptin (anti-Her-2 neu antibody), Rituxan®, Zevalin, Bevacizumab (Avastin), Bexxar, Campathg®, Oncolym, 17-1A (Edrecolomab), 3F8 (anti-neuroblastoma antibody), MDX-CTLA4, IMC-C225 (Cetuximab) and Mylotarg.

Additional examples of immune enhancing agents and treatments include immune cells such as lymphocytes, plasma cells, macrophages, dendritic cells, NK cells and B-cells that either express antibody against the cell proliferative disorder or otherwise are likely to mount an immune response against the cell proliferative disorder. Cytokines that enhance or stimulate immunogenicity include IL-2, IL-1α, IL-1β, IL-3, IL-6, IL-7, granulocyte-macrophage-colony stimulating factor (GMCSF), IFN-γ, IL-12, TNF-α, and TNFβ, which are also non-limiting examples of immune enhancing agents. Chemokines including MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, PARC, TARC, LARC/MIP-3α, CKβ, CKβ6, CKβ7, CKβ8, CKβ9, CKβ11, CKβ12, C10, IL-8, ENA-78, GROα, GROβ, GCP-2, PBP/CTAPIIIβ-TG/NAP-2, Mig, PBSF/SDF-1, and lymphotactin are further non-limiting examples of immune enhancing agents.

The invention further provides kits, including agents, nucleic acids proteins, and pharmaceutical formulations, packaged into suitable packaging material, optionally in combination with instructions for using the kit components, e.g., instructions for performing a method of the invention. In one embodiment, a kit includes an amount of an agent that increases expression or activity of a glycogenic enzyme, and instructions for administering the agent to a subject in need of treatment on a label or packaging insert. In another embodiment, a kit includes an amount of an agent that decreases expression or activity of a glycogenolytic enzyme, and instructions for administering the agent to a subject in need of treatment on a label or packaging insert. In yet another embodiment, a kit includes an amount of an agent that increases accumulation of intracellular glycogen, and instructions for administering the agent to a subject in need of treatment on a label or packaging insert. In additional aspects, a kit further includes an anti-tumor or immune enhancing agent, for example, an alkylating agent, anti-metabolite, plant alkaloid, plant extract, antibiotic, nitrosourea, hormone, nucleoside analogue, nucleotide analogue, or antibody. In still further aspects, a kit includes an article of manufacture, for delivering the agent into a subject locally, regionally or systemically, for example.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a cell prolferative disorder, an assay for identifying an agent having anti-cell proliferative activity, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, invention pharmaceutical compositions can be included in a container, pack, or dispenser together with instructions for administration to a subject to treat a cell proliferative disorder such as a tumor or cancer. Instructions may additionally include indications of a satisfactory clinical endpoint or any adverse symptoms that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a protein/nucleic acid stabilizing agent. The kit can also include control components for assaying for activity, e.g., a control sample or a standard. Each component of the kit can be enclosed within an individual container or in a mixture and all of the various containers can be within single or multiple packages.

The proteins, nucleic acids, agents and other compositions and methods of the invention can further employ pharmaceutical formulations. Such pharmaceutical formulations are useful for administration to a subject in vivo or ex vivo.

Pharmaceutical formulations include “pharmaceutically acceptable” and “physiologically acceptable” carriers, diluents or excipients. As used herein the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions.

Pharmaceutical formulations can be made to be compatible with a particular local, regional or systemic route of administration. Thus, pharmaceutical formulations include carriers, diluents, or excipients suitable for administration by particular routes. Specific non-limiting examples of routes of administration for compositions of the invention are parenteral, e.g., intravenous, intradermal, intramuscular, subcutaneous, oral, transdermal (topical), transmucosal, intra-cranial, intra-ocular, rectal administration, and any other formulation suitable for the administration protocol or condition to be treated. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride can be included in the composition. Including an agent which delays absorption, for example, aluminum monostearate or gelatin can prolong absorption of injectable compositions.

Sterile injectable formulations can be prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

The pharmaceutical formulations can be prepared with carriers that protect against rapid elimination from the body, such as a controlled release formulation or a time delay material such as glyceryl monostearate or glyceryl stearate. The formulations can also be delivered using articles of manufacture such as implants and microencapsulated delivery systems to achieve local, regional or systemic sustained delivery or controlled release.

Biodegradable, biocompatable polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to known methods, for example, as described in U.S. Pat. No. 4,522,811.

Additional pharmaceutical formulations appropriate for administration are known in the art (see, e.g., Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins Publishers (1999); Kibbe (ed.), Handbook of Pharmaceutical Excipients American Pharmaceutical Association, 3^{rd} ed. (2000); and Remington's Pharmaceutical Principles of Solid Dosage Forms, Technonic Publishing Co., Inc., Lancaster, Pa., (1993)).

The compositions used in accordance with the invention, including nucleic acids, proteins, agents, treatments and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired therapeutic effect. The unit dosage forms will depend on a variety of factors including, but not necessarily limited to, the particular composition employed and the effect to be achieved, and the pharmacodynamics and pharmacogenomics of the subject to be treated.

The invention provides cell-free (e.g., in solution, in solid phase) and cell-based (e.g., in vitro or in vivo) methods of identifying and screening for agents and treatments having anti-cell proliferative activity and useful for treating cell proliferative disorders (e.g., cancers and tumors). The methods can be performed in solution, in vitro using prokaryotic or eukaryotic cells, and in vivo, for example, using a disease animal model. The agents and treatments identified as capable of increasing glycogen levels, for example, to toxic levels, can be used alone or in combination with gene transfer in order to decrease expression or activity of a protein that participates in metabolism, catabolism, degradation or removal of glycogen (e.g., a glycogenic enzyme), or to increase or stimulate expression or activity of a protein that participates in synthesis or accumulation of glycogen (e.g., a glycogenolytic enzyme or glucose transporter).

In one embodiment, a method of identifying an agent having anti-cell proliferative activity includes: contacting a cell that produces glycogen with a test agent; and assaying for glycogen toxicity in the presence of the test agent or following contacting with the test agent. Glycogen toxicity identifies the test agent as an agent having anti-cell proliferative activity. In another embodiment, a method of identifying an agent having anti-cell proliferative activity includes: contacting a cell that produces glycogen with a test agent; and assaying for cell viability in the presence of the test agent or following contacting with the test agent. Reduced or decreased cell viability identifies the test agent as an agent having anti-cell proliferative activity.

Cell-based screening assays of the invention can be practiced by using non-transformed cells that produce glycogen or exhibit glycogen toxicity. Such cells will typically express one or more glycogenic or glycogenolytic enzymes, whose expression or activity can be assayed in order to identify agents having anti-cell prolieferative activity.

Thus, in yet another embodiment, a method of identifying an agent having anti-cell proliferative activity includes: contacting a cell that expresses a glycogenic enzyme or a glycogenolytic enzyme with a test agent; and measuring activity or expression of the glycogenic enzyme or glycogenolytic enzyme in the presence of the test agent or following contacting with the test agent. Increased or decreased expression or activity of the glycogenic enzyme or glycogenolytic enzyme, respectively, identifies the test agent as an agent having anti-cell proliferative activity. In various aspects, one or more glycogenic enzymes such as glycogenin, glycogenin-2, glycogen synthase, glycogenin interacting protein (GNIP), protein phosphatase 1 (PP-1), glucose transporter (GLUT), a glycogen targeting subunit of PP-1 isoform or family member (e.g., G_{L} (PPP1R₃B, PPP1R4), PTG (PPP1R₃C, PPP1R₅), PPP1R3D (PPP1R6) or Gₘ/R_{G1} (PPP1R3A, PPP1R3)), a hexokinase isoform or family member, glutamine-fructose-6-phosphate transaminase, or one or more glycogenolytic enzymes, such as glycogen phosphorylase, debranching enzyme, phosphorylase kinase, glucose-6-phosphatase, PPP1R1A (protein phosphatase 1, regulatory Inhibitor subunit 1A), PPP1R2 (protein phosphatase 1, regulatory subunit 2), phosphofructokinase, a glycogen synthase kinase-3 isoform, GCKR glucokinase regulatory protein or α-glucosidase, are measured.

Alternatively, transformed cells (e.g., with a nucleic acid sequence) can be employed in the screening methods. For example, a cell can be stably or transiently transformed with a gene whose expression is modulated by a regulatory region of a glycogenic enzyme or glycogenolytic enzyme, and changes in expression of the gene can indicate whether the agent has anti-cell proliferative activity. Particular examples are cells transformed with a reporter gene, which refers to a gene encoding a protein that can be detected, such as galactosidase, chloramphenicol acetyl transferase, glucose oxidase, luciferase, or green fluorescent protein. Expression can be modulated by a promoter selected from glycogenin, glycogenin-2, glycogen synthase, glycogenin interacting protein (GNIP), protein phosphatase 1 (PP-1), glucose transporter (GLUT), a glycogen targeting subunit of PP-1 family, a hexokinase family member, glutamine-fructose-6-phosphate transaminase, glycogen phosphorylase, debranching enzyme, phosphorylase kinase, glucose-6-phosphatase, PPP1R1A (protein phosphatase 1, regulatory Inhibitor subunit 1A), PPP 1 R2 (protein phosphatase 1, regulatory subunit 2), phosphofructokinase, a glycogen synthase kinase-3 isoform, GCKR glucokinase regulatory protein or α-glucosidase, for example.

Thus, in still another embodiment, a method of identifying an agent having anti-cell proliferative activity includes: contacting a cell that expresses a gene whose expression is modulated by a regulatory region of a glycogenic enzyme or a glycogenolytic enzyme with a test agent; and measuring expression of the gene in the presence of the test agent or following contacting with the test agent, wherein increased or decreased expression of the gene identifies the test agent as an agent having anti-cell proliferative activity.

Particular non-limiting examples of cell types useful in practicing the screening methods include cells from any tissue or organ that is susceptible to a cell proliferative disorder. For example, cells include hyperproliferative, immortalized, tumor and cancer cell lines and primary isolates derived from brain, head or neck, breast, esophagus, mouth, stomach, lung, gastrointestinal tract, liver, pancreas, kidney, adrenal gland, bladder, colon, rectum, prostate, uterus, cervix, ovary, testes, skin, muscle or hematopoetic system.

In a further embodiment, a method of identifying an agent having anti-cell proliferative activity includes: providing a test agent that increases expression or activity of a glycogenic enzyme; contacting a cell that expresses a glycogenic enzyme with the test agent; and assaying for glycogen toxicity in the presence of the test agent or following contacting with the test agent. Glycogen toxicity identifies the test agent as an agent having anti-cell proliferative activity.

In an additional embodiment, a method of identifying an agent having anti-cell proliferative activity includes: providing a test agent that binds to a glycogenic or a glycogenolytic enzyme; contacting a cell that expresses a glycogenic or a glycogenolytic enzyme with the test agent; and assaying for glycogen toxicity in the presence of the test agent or following contacting with the test agent. Glycogen toxicity identifies the test agent as an agent having anti-cell proliferative activity.

In an auxiliary embodiment, a method of identifying an agent having anti-cell proliferative activity includes: providing a test agent that decreases expression or activity of a glycogenolytic enzyme; contacting a cell that expresses a glycogenolytic enzyme with the test agent; and assaying for glycogen toxicity in the presence of the test agent or following contacting with the test agent. The glycogen toxicity identifies the test agent as an agent having anti-cell proliferative activity.

In a still further embodiment, a method of identifying an agent having anti-cell proliferative activity includes: contacting a glycogenic enzyme or a glycogenolytic enzyme with a test agent; and measuring activity of the glycogenic enzyme or glycogenolytic enzyme in the presence of the test agent or following contacting with the test agent. Increased or decreased activity of the glycogenic enzyme or glycogenolytic enzyme, respectively, identifies the test agent as an agent having anti-cell proliferative activity. In various aspects, the contacting is in a cell-free system (e.g., in solution or in solid phase), or in a cell-based system (e.g., in vitro or in vivo).

The term “contacting,” when used in reference to an agent or treatment, means a direct or indirect interaction between the agent and the other referenced entity. A particular example of direct interaction is binding. A particular example of an indirect interaction is where the agent acts upon an intermediary molecule which in turn acts upon the referenced entity. Thus, for example, contacting a glycogenic enzyme or a glycogenolytic enzyme with a test agent includes allowing the agent to bind to the enzyme, or allowing the agent to act upon an intermediary that in turn acts upon the enzyme.

The terms “measuring” and “assaying,” and grammatical variations thereof are used interchangeably herein and refer to either qualitative and quantitative determinations, or both qualitative and quantitative determinations. When the terms are used in reference to glycogen levels, glycogen or cell toxicity or expression or activity of an enzyme (e.g., a glycogenic or a glycogenolytic enzyme), and so forth, any means of assessing glycogen levels, toxicity or expression or activity of an enzyme, etc. are contemplated, including the various methods set forth herein and otherwise known in the art. For example, glycogen toxicity can be assayed by screening for one or more morphological changes associated with glycogen toxicity; screening for cell viability; screening for inhibition or reduction of cell proliferation, growth or survival.

Test agents and treatments can be applied to any prokaryotic or eukaryotic cell in which glycogen can be measured or whose growth, proliferation or viability can be measured. For example, immortalized, hyperproliferative or tumor or cancer cells can be grown in culture under conditions and for a time sufficient to allow contact and measurement or detection of glycogen accumulation, glycogen toxicity or reduced cell growth, proliferation, survival or viability.

Glycogen accumulation can be detected by a variety of ways known in the art. An exemplary method is described in Example 1, which involves glucoamylase-mediated hydrolysis of glycogen to glucose followed by colorimetric quantitation. The values are expressed as micrograms of reduced glucose per million cells. A high-throughput screening assay that measures glucose incorporation into glycogen has been developed (Berger J, Hayes N S. Anal Biochem. 1998 Aug. 1;261 (2): 159) and can be used to measure glycogen accumulation for the purpose of identifying agents and treatments that increase or stimulate intracellular glycogen accumulation. http://neo.pharm.hiroshima-u.ac.jp/ccab/2nd/mini_review/mr132/yano.html

Histological analysis can also be used to detect glycogen. For example, glycogen can be observed in histological sections using the McMannus' Periodic Acid Schiff (PAS) stain. The stain is a histochemical reaction in that the periodic acid oxidizes the carbon to carbon bond forming aldehydes which react to the fuchsin-sulfurous acid which form the magenta color. Alternatively, a monoclonal antibody that binds glycogen in combination with immuno-gold particles can detect glycogen using, for example, an electron microscope (Baba, O. Kokubyo Gakkai Zasshi. 60:264 (1993)).

Glycogen content can be determined either directly or indirectly. For example, incorporating radio-labeled-glucose, such as [¹³C or ¹⁴C]-glucose, into glycogen followed by radiographic quantitation. An alternative approach to determine glycogen is by hydrolysis to glucose monomers using glucoamylase and measuring reduced glucose colorimetrically, for example, with glucose Trinder colorometric reagent (Sigma, St. Louis, Mo.) (Kepler and Decker. In: Methods of Enzymatic Analysis, Eds. H. U. Bergenmeyer and K. Gawehn, Academic Press, New York, 4:1127-1131(1974). Another alternative assay for glycogen is colorimetric detection of acid-reduced glucose with anthrone reagent, (Lab Express, Inc. Fairfield, N.J.) (Seifter et al., Arch. Biochem. 25:191 (1950). These assays can also be formatted for high throughput screening of agents and treatments that increase or stimulate intracellular glycogen accumulation.

Glycogen levels can also be determined in vivo. For example, Fourier Transform Infrared Spectroscopy has been used to determine glycogen levels in human tissues (Yano K., Evaluation Of Glycogen Levels In Human Carcinoma Tissues By Fourier Transform Infrared Spectroscopy. “Trends in Analytical Life Sciences” Vol.1 (CCAB97) Cyber Congress on Analytical BioSciences held on Internet Aug. 21, 1997). NMR spectroscopy is a non-invasive means to study muscle glycogen metabolism continuously in vivo (Roden and Shulman, Annu Rev Med. 50:277 (1999)).

Cell toxicity can be measured in a variety of ways on the basis of calorimetric, luminescent, radiometric, or fluorometric assays known in the art. Colorimetric techniques for determining cell viability include, for example, Trypan Blue exclusion (see, for example, Examples 1 and 2). In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in the cell's lysosomes; viable cells can be determined with a light microscope by quantitating numbers of Neutral Red stained cells. Tetrazolium salts (e.g. MTT, XTT, WST-1) are useful for quantitating cell viability in a colorimetric assay format (Roche Diagnostics Corp. Indianapolis, Ind.). Tetrazolium salts are cleaved to formazan by the “succinate-tetrazolium reductase” system in the respiratory chain of the mitochondria, which is only active in metabolically intact cells.

Fluorometric techniques for determining cell viability include, for example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. The Alamar Blue assay (Alamar Biosciences Inc Sacramento Calif.) incorporates a redox indicator that changes color or fluorescence in response to metabolic activity and is used to quantitate viability or proliferation of mammalian cells. Alamar Blue can be measured spectrophotometrically (fluorescence). Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochrome-labeled antibody. The fluorecencent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for determining cell proliferation include, for example, [³H]-Thymidine, which is incorporated into newly synthesized DNA of living cells and frequently used to determine proliferation of cells. Chromium (⁵¹Cr)-release from dead cells can be quantitated by scintillation counting in order to quantitate cell viability.

Luminecent techniques for determining cell viability include, for example, the CellTiter-Glo luminescent cell viability assay (Promega Madison Wis.). This technique quantifies the amount of ATP present to determine the number of viable cells.

Commercially available kits for determining cell viability and cell proliferation include, for example, Cell Proliferation Biotrak ELISA (Amersham Biosciences Piscataway, N.J.); the Guava ViaCount™ Assay, which provides rapid cell counts and viability determination based on differential uptake of fluorescent reagents (Guava Technologies, Hayward, Calif.); the CyQUANT® Cell Proliferation Assay Kit (Molecular Probes, Inc., Eugene, Oreg.); and the CytoLux Assay Kit (PerkinElmer Life Sciences Inc., Boston, Mass.). The DELFIA® Assay Kits (PerkinElmer Life Sciences Inc., Boston, Mass.) can determine cell proliferation and toxicity using a time-resolved fluorometric method. BRET2 (Bioluminescence Resonance Energy Transfer) is an advanced, non-destructive, assay technology designed to monitor protein-protein interactions and intracellular signaling events in live cells (PerkinElmer Life Sciences Inc., Boston, Mass.). BRET2 is based upon the transfer of resonant energy from a bioluminescent donor protein to a fluorescent acceptor protein using Renilla luciferase (Rluc) as the donor and a mutant of the Green Fluorescent Protein (GFP 2) as the acceptor molecule. BRET2 is analogous to fluorescence resonance energy transfer (FRET), but eliminates the need for an excitation light source and its associated problems (e.g. high background caused by autofluorescence). Cell Death Detection ELISA is a photometric enzyme immunoassay for quantitative in vitro determination of cytoplasmic histone-associated DNA fragments (mono- and oligonucleosomes) after cell death (Roche Diagnostics Corp., Indianapolis, Ind.). The LDH Cytotoxicity Detection Kit measures lactate dehydrogenase (LDH) released from damaged cells (Takara.Mirus.Bio, Madison, Wis.). The Quantos™ Cell Proliferation Assay is a fluorescence-based assay that measures the fluorescence of a DNA-dye complex from lysed cells (Stratagene, La Jolla, Calif.). The CellTiter-Glo cell viability assay is a luminescent assay for measuring cell viability (Promega, Madison Wis.).

Test agents and treatments are available or can be produced using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other approaches are applicable to peptide, non-peptide oligomer or small molecule libraries of compounds.

Methods for the synthesis of molecular libraries are known in the art (see, e.g., DeWitt et al., Proc. Natl. Acad, Sci. U.S.A. 90:6909 (1993); Erb et al., Proc. Natl. Acad. Sci. U.S.A. 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int Ed. Engl. 33:2061 (1994); and Gallop et al., J Med. Chem. 37:1233 (1994)). The libraries of compounds may be presented in solution (e.g., Houghten, Biotechniques 13:412 (1992)), or beads (Lam, Nature 354:82 (1991)), on chips (Fodor Nature 364:555 (1993)), bacteria (U.S. Pat. No. 5,223,409), spores (U.S. Pat. No. 5,233,409), plasmids (Cull et al., Proc. Natl. Acad. Sci. USA 89:1865 (1992)) or on phage (Scott and Smith, Science 249:386 (1990); Devli Science 249:404 (1990); Cwirla et al., Proc. Natl. Acad. Sci. U.S.A. 87:6378 (1990); Felici, J. Mol. Biol. 222:301 (1991); and U.S. Pat. No. 5,233,409).

As an example of an in vitro assay for identifying agents or treatments that increase glycogen in cells, cells can be grown in tissue culture microtitre plates. These microtitre plates may be in any form suitable for measuring glycogen accumulation or cell toxicity. In order to conduct the assay, cell lines (e.g., cancer cell lines) can be seeded onto the plates under conditions suitable for growth of the cell line and at an appropriate cell density. The test agent can be applied to the cells at a variety of concentrations and in a variety of formulations either manually or in an automated fashion, for example, using a robotic apparatus. Alternatively, the cells can be subjected to the test treatment, for example, alterations in temperature, pH, oxygenation (e.g., hypoxia), salt or ion concentration, etc. Determining cell glycogen accumulation or cell toxicity will be dictated by the specific assay employed, as described herein or otherwise known in the art. For example, a luminometer would be used to determine results from luminescent-based assays, a fluorimeter or flow cytometer would be used to quantitate fluorescent-based assays, a scintillation counter would be used to determine results from a radiometric-based assay, etc.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All publications, patents, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to “a gene or nucleic acid” includes a plurality of genes or nucleic acids and reference to “a cell” can include reference to all or a part of a cell or plurality of cells, and so forth.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes various exemplary materials and methods.

Recombinant Adenovirus Vectors: Synthetic oligonucleotides were designed to amplify the open reading frame of the human G_{L} cDNA (SEQ ID NO:3- GenBank Accession number XM_{—}015545, positions 10 bp to 1183 bp) (SEQ ID NO:1-sense primer GACCAATTGTCGCGCTTGCCACAACC; SEQ ID NO:2-anti-sense primer CTGCTCGAGCGCGCCAGCCACCACT). A 1192 bp fragment was amplified using the polymerase chain reaction (PCR) from a human fetal liver Marathon cDNA library (Clontech, Inc.). This fragment was subcloned into the EcoRV site of pBluescript (Stratagene, Inc.) creating pSSBS-G_{L}. This plasmid was sequenced with threefold coverage. The HincII fragment of PSSBS-G_{L} containing the human G_{L} cDNA was subcloned into the PmeI sites of pShuttle from the Adeno-X Expression System (Clontech, Inc.) to create pShuttle-hsG_{L}.

The translational enhancer element in the 5′ untranslated region of heat shock protein 70 (SEQ ID NO:6-From Genbank Accession number AC020768 corresponding to M11717, positions 276 bp to 488 bp) was amplified by PCR (SEQ ID NO:4-sense primer GGCAATTGAACGGCTAGCCTGAGGAGCTGC; SEQ ID NO:5-anti-sense primer CCACTAGTGCGGTTCCCTGCTCTCTGTCG) and a 213 bp fragment was subcloned into the SmaI site of the pNEB 193 vector (New England Biolabs, Inc.). The resulting clone was sequenced with threefold coverage. A 257 bp XbaI/SpeI fragment was blunt cloned into the unique NheI site 5′ of the G_{L} cDNA in pShuttle-G_{L} to create pShuttle-hspG_{L}.

The transcriptional enhancer element (WPRE) in the Woodchuck hepatitis B virus (SEQ ID NO 9: Genbank Accession number J02442, positions 1093 bp to 1714 bp) was amplified by PCR (SEQ ID NO:7-sense primer TCGGGATCCAATCAACCTCTGGATTACA; SEQ ID NO:8-anti-sense primer TGCTCTAGACAAGCAACACGGACC) and a 641 bp fragment was subcloned using the pGEM-T vector system (Promega, Inc.). The resulting clone was sequenced with threefold coverage. A NotI/XbaI restriction fragment containing the WPRE element was cloned 3′ of the G_{L} cDNA to create pShuttle-G_{L} WPRE.

Clontech's Adeno-X Expression System was used to create the adenovirus vectors used for this study. Transferring the empty pShuttle vector, pShuttle-G_{L}, pShuttle-hspG_{L}, and pShuttle-G_{L} WPRE into the Adeno-X adenovirus genome according to the manufacturer's instructions created the recombinant adenovirus vectors AdpSh, AdG_{L}. AdhspG_{L}, and AdG_{L} WPRE, respectively. Transfecting HEK 293 cells with the adenovirus vector DNA according to Clontech's Adeno-X Expression System instructions produced crude adenovirus stocks. Adenovirus particles were purified using the Adenopure Adenovirus Purification kit (Puresyn, Inc.) according to the manufacturer's instructions.

Cell Culture: HeLa (human cervical epithelial adenocarcinoma), MCF7 (human breast epithelial adenocarcinoma) and LoVo (human colorectal epithelial adenocarcinoma) cell lines were obtained from the American Type Culture Collection (ATCC, Rockville, Md.). HeLa and MCF7 cells were cultured in high-glucose Dulbecco's Minimal Essential Medium (DMEM, Gibco #12800-017) supplemented with 5% heat-inactivated fetal bovine serum (FBS), 2 mM glutamine, penicillin (100 U/ml)-streptomycin (100 ug/mg), and 2.2 g/liter of NaHCO₃. LoVo cells were cultured in Kaighn's Modification of Ham's F-12 medium (F-12K, ATCC) supplemented with 5% heat-inactivated FBS, 2 mM glutamine, penicillin (100 U/ml), streptomycin (100 ug/mg), and 2.2 g/liter of NaHCO₃.

Cultured cells lines were seeded on six or twelve well tissue culture plates. When they reached 70-85% confluence, cells were infected with various amounts of recombinant G_{L} adenovirus or control adenovirus. Adenovirus was added to each well in 300 μl medium and incubated for 2 hours at 37° C., 5% CO₂. After incubation, 1.5 ml of medium was added and incubated at 37° C., 5% CO₂. The medium was changed every day. At various time points post-infection, viable cells were counted using Trypan Blue and the remaining cells were collected and frozen for subsequent glycogen measurements.

Trypan Blue Viability Cell Counts: Trypan Blue (0.4%, Gibco) was used to stain dead and dying cells. Cells were removed from the tissue culture plates with 0.25% Trypsin-EDTA (Gibco #25200-056) and resuspended in 1 ml phosphate buffered saline (PBS). Manual cell counts were performed with a Neubauer hemocytometer.

Glycogen Assay: Enzymatic glycogen hydrolysis to glucose was performed according to Keppler and Decker with some modifications (Keppler and Decker, 1984 in: Methods of Enzymatic Analysis, 3^{rd} ed. (Bergmeyer, H. U. Bergmeyer, J., and Grab, M. Eds.), Vol. 6, pp. 11-18, VCH, New York.). In brief, frozen cell pellets were subjected to three rounds of freezing and thawing to disrupt cell membranes. Cell pellets were resuspended in 200 μl 250 mU glucoamylase in 0.2 M sodium acetate buffer, pH 4.8. Lysates were incubated for two hours at 45° C. with shaking. Lysates were cleared by centrifugation at 2500 rpm for 10 min. Supernatants (5 μl) and glucose standards were transferred to a 96-well plate and neutralized with 10 μl of 0.25N sodium hydroxide. Glucose was then determined with the glucose Trinder colorometric reagent (Sigma, 315-500). The intensity of the color reaction was measured at 505nm using a Molecular Devices VERSAmax microplate reader.

Roscovitine Studies: Roscovitine (Calbiochem #557362), [2-(R)-(1-ethyl-2-hydroxyethylamino)-6-benzyl amino-9-isopropylpurine], is a potent and selective inhibitor of the cyclin-dependent kinases Cdk2 and Cdc2. Roscovitine stock solution was prepared in dimethylsulfoxide (DMSO) and stored at −20° C. until use. The drug was diluted in medium and used at final concentration of 35 μM. In all cases, untreated cells behaved identically to those treated with DMSO alone. Roscovitine was added to the cell cultures 24 hours after transduction with adenovirus, and incubated for 48 to 72 hours. Cells were then collected for Trypan Blue viability counts and glycogen measurement.

### Example 2

This example describes data indicating that transferring a gene encoding a protein that increases intracellular glycogen into a cell can increase glycogen to levels that are toxic to the cell.

A nucleic acid encoding a member of the glycogen targeting subunit family that targets PP-1 to glycogen particles was cloned into a recombinant adenovirus vector for expression in target human cancer cell lines. The cDNA encoding the wildtype human G_{L} protein was cloned into an adenovirus vector as described in Example 1. The recombinant adenovirus vector expressing G_{L} cDNA was designated AdG_{L}. As adenovirus itself can be toxic to cells at high doses, a control vector identical to AdG_{L} but lacking G_{L} cDNA was manufactured and designated AdpSh.

High titre adenovirus particles produced from the recombinant viral vectors were used to infect various human cancer cell lines as described in Example 1. In brief, human cervical epithelial adenocarcinoma cell line (HeLa) was cultured to a confluency of approximately 70% and then infected with either AdG_{L}, or control AdpSh. After 24 hours morphological changes were observed in AdG_{L}-infected cells compared to AdpSh-treated cells. In addition, AdG_{L}-infected cells were larger in size and an increase in cell rounding was observed.

Cell viability after viral infection was assessed using the Trypan Blue exclusion assay. Cells stained with Trypan Blue and viable cells are counted using a Neubauer hemocytometer. Viable cells exclude the dye whereas dead and dying cells take up the blue dye and can easily be distinguished under a light microscope.

Seventy two hours after infection, significant Trypan Blue uptake was observed in AdG_{L}-treated cells in comparison to control AdpSh-treated cells. Thus, overexpression of G_{L} induced death in the HeLa cell line.

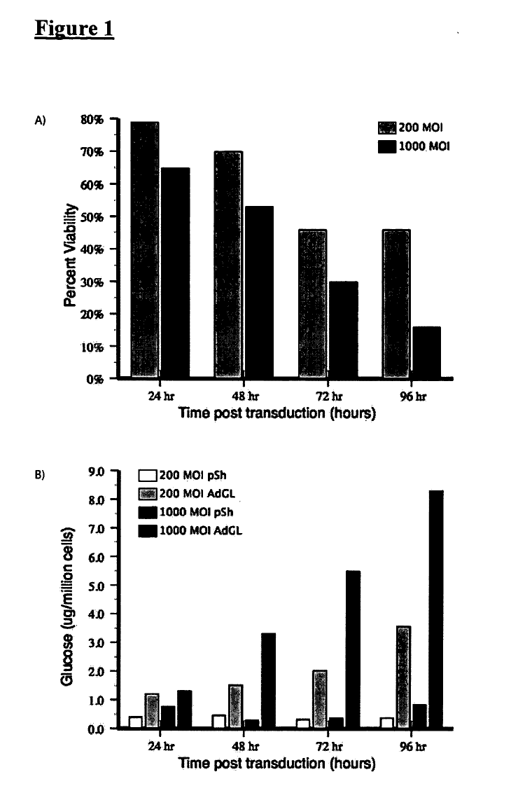

The observed reduction in cell viability and increased glycogen after infection of HeLa cells with AdG_{L} is time and viral dose dependant. HeLa cells were infected at either 200 multiplicity of infection (MOI) or 1000 MOI of either AdG_{L} or AdpSh adenovirus (FIG. 1). The ratio of counts of viable cells that exclude Trypan Blue from AdG_{L}-infected cells to that of control AdpSh-infected cells is expressed as a percentage (FIG. 1, Panel A). The results indicate that viability of AdG_{L}-infected cells is reduced over time.

Increasing the dose of virus also results in reduced viability of AdG_{L}-infected cells. A dose-dependent increase in glucose derived from glucoamylase-reduced glycogen with increasing multiplicity of infection (MOI) from 200 to 1000 was observed in cells infected with AdG_{L} (FIG. 1, Panel B). In contrast, infection of cells with control AdpSh resulted in minimal nonspecific accumulation of glucose derived from glucoamylase-reduced glycogen at either multiplicity of infection (FIG. 1, Panel B).

The results indicate that increased glycogen accumulation correlated with decreased cell viability. These findings corroborate that the accumulation of glycogen induced by the overexpression of G_{L} results in cell death.

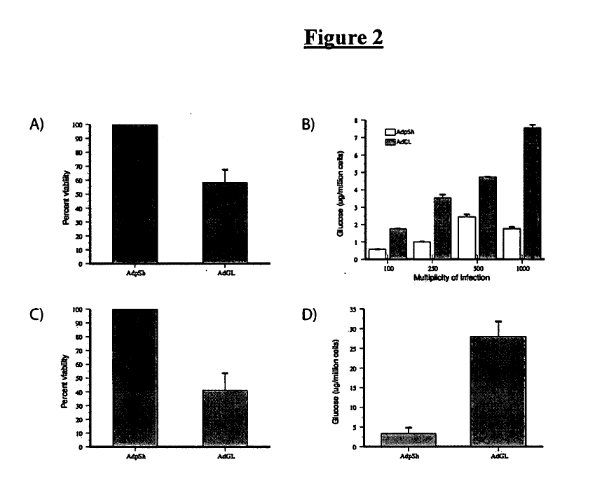

To confirm the applicability of this strategy to hyperproliferative cancer cells in general, the AdG_{L} adenovirus was used to infect two additional cell lines as described in Example 1. Overexpression of G_{L} in a human breast epithelial adenocarcinoma (MCF7) and human colorectal epithelial adenocarcinoma (LoVo) resulted in similar reductions in cell viability and increases in glucose derived from glucoamylase-reduced glycogen to that of the HeLa cell line (FIG. 2). These data confirm that the accumulation of glycogen induced by adenovirus expressing G_{L} is able to kill cancer cells generally.

### Example 3

This example describes data indicating that transferring a gene encoding a protein that increases intracellular glycogen in a cell, in combination with a drug that inhibits cell growth, enhances glycogen accumulation and death of the cell.

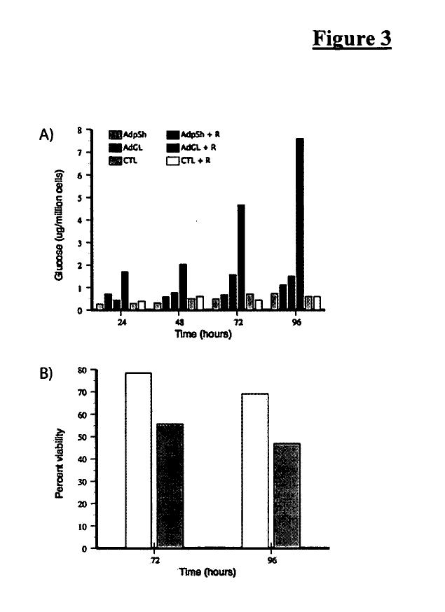

Roscovitine is a potent and selective inhibitor of the cyclin-dependent kinase Cdk2 and Cdc2 and is cytostatic. To study whether this drug would lead to enhanced accumulation of glycogen and a corresponding decrease in cell viability when used in combination with AdG_{L}, HeLa cells were infected with AdG_{L} or AdpSh adenovirus (500 MOI) followed by addition of 35 μM roscovitine (FIG. 3).

The combination of AdG_{L} and roscovitine significantly increased the amount of glycogen in infected HeLa cells. In contrast, uninfected control cells showed no significant increases in glycogen accumulation with or without roscovitine (FIG. 3, Panel A). The ratio of counts of viable cells that exclude Trypan Blue from AdG_{L}-infected cells to that of control AdpSh-infected cells is expressed as a percentage for both roscovitine-treated and untreated cells (FIG. 3, Panel B).

The data therefore demonstrate that a compound which inhibits, reduces or prevents growth of cancer cells can be used in combination with a vector expressing a glycogenic enzyme (e.g., adenoviral G_{L}) to increase glycogen to levels that are toxic to cancer cells. Moreover, amounts of glycogen achieved are enhanced relative to expressing a glycogenic enzyme alone in the target cells.

### Example 4

This example describes data indicating that modifications can be made to gene transfer vectors in order to increase levels of expression of the gene.

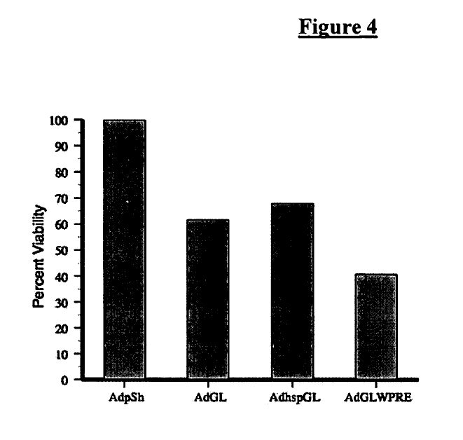

To increase the level of expression of the G_{L} cDNA, two nucleic acid enhancing elements were compared with the AdG_{L} vector in their ability to decrease cell viability (FIG. 4). The first element increases efficiency of mRNA translation and was originally identified in the 5′ untranslated region (5′ UTR) of the human heat shock protein-70 (hsp70) gene (Vivinus et al., Eur J Biochem. 268:1908 (2001)). This element was incorporated into the AdG_{L} vector thus creating AdhspG_{L} as described in Example 1. The second element, termed WPRE, was identified in the Woodchuck Hepatitis virus and is a cis-acting RNA posttranscriptional regulatory element (Donello et al., J Virol. 72:5085 (1998)). WPRE was incorporated into the AdG_{L} vector thus creating AdG_{L} WPRE as described in Example 1.

HeLa cells were individually infected with each viral vector (500 MOI) as previously described. The ratio of counts of viable cells that exclude Trypan Blue from virus-infected cells to that of control AdpSh-infected cells is expressed as a percentage. The hsp70 5′ UTR element did not significantly decrease the viability of infected HeLa cells. Incorporation of the WPRE element resulted in an approximately 1.5 fold reduction in cell viability compared to AdG_{L} alone. Thus, genetic modifications to the gene transfer vector can increase expression of the gene of interest, for example, G_{L}, thereby enhancing glycogen accumulation, and in turn, reducing cell viability.

### Example 5

This example describes several exemplary alpha-glucosidase activity assays to identify inhibitory agents.

In brief, 10 ul of an test agent solution and 990 ul of a substrate solution (10 mM maltose) is added to an end-capped mini-column containing alpha-glucosidase immobilized Sepharose (10 mg-wet gel). The assay is initiated by adding 1.0 ml of a model intestinal fluid containing 10 mM maltose. After incubation at 37 degree Celsius for 30 min, liberated glucose is quantitated by Glucose CII-Test (Wako Pure Chemical Co., Japan). The inhibitory activity is calculated based on the difference in the amount of glucose in the filtrate with or without the test agent. The amount of the test agent that inhibits 50% of alpha-glucosidase activity under the assay conditions is defined as the IC₅₀ (Matsumoto et al., Analytical Sciences, 18:1315 (2002)).

Additional exemplary assays for identifying agents that inhibit alpha-glucosidase is to test agents against alpha-glucosidase (yeast, type I, produced by Sigma Chemical Co.) as well as maltase and saccharase prepared from porcine intestinal mucosa (prepared as described in Borgstrom and Dahlgvist in Acta Chem. Scand., 12:1997 (1958)). When maltose and sucrose are used as a substrate, 0.25 ml of alpha-glucosidase solution prepared by diluting with 0.02M phosphate buffer (pH 6.8) is mixed with 0.5 ml of a solution of a test agent in the same buffer, and 0.25 ml of 0.05M maltose or 0.05M sucrose as the substrate in the same buffer. The mixture is allowed to react at 37 degree C. for 10 minutes. Glucose B-Test Reagent (3 ml; which is a glucose oxidase reagent for glucose measurement, Wako Pure Chemical Co., Japan) is then added and the mixture warmed at 37 degree C. for 20 minutes. The absorbance of the reaction solution is subsequently measured at 505 nm.

The inhibitory activity of test agents against alpha-glucosidase (yeast, type I, Sigma Chemical Co.) and glucoamylase (Rhizopus mold, Sigma Chemical Co.), when p-nitrophenyl-alpha-D-glucopyranosidase is used as a substrate, is determined by adding to 0.25 ml of 0.02M phosphate buffer (pH 6.8) containing 0.005 mg/ml of alpha-glucosidase 0.5 ml of a test agent solution in the same buffer and 0.25 ml of a solution of 0.01M p-nitrophenyl-alpha-D-glucopyranosidase in the same buffer, and allowing the mixture to react at 37.degree C. for 15 minutes. Sodium carbonate solution (3 ml, 0.1M) is added to terminate the reaction, and the absorbance is measured at 400 nm. The 50% inhibition concentration is calculated from the inhibition rates (%) which are determined by three to five different concentrations of the test agent.

1. A method of increasing glycogen to toxic levels in a cell, comprising expressing in the cell a gene product that increases the amount of glycogen to toxic levels in the cell.

2. The method of claim 1, wherein the gene product comprises a protein that increases synthesis or intracellular accumulation of glycogen.

3. The method of claim 1, wherein the gene product comprises a protein that decreases glycogen metabolism, catabolism, utilization, degradation or removal.

4. The method of claim 1, wherein the glycogen is in an amount that causes a morphological change associated with glycogen toxicity.

5. The method of claim 4, wherein the morphological change associated with glycogen toxicity comprises cell swelling, increased numbers of lysosomes, increased size of lysosomes, or a structural change in lysosomes.

6. The method of claim 1, wherein the glycogen is in an amount that causes lysis or apoptosis of the cell.

7. The method of claim 1, wherein the glycogen is in an amount that inhibits or reduces proliferation, growth or survival of the cell.

8. The method of claim 1, wherein the gene product comprises a glycogenic enzyme.

9. The method of claim 1, wherein the glycogenic enzyme comprises glycogenin, glycogenin-2, glycogen synthase, glycogenin interacting protein (GNIP), protein phosphatase 1 (PP-1), glucose transporter (GLUT), a glycogen targeting subunit of PP-1 isoform or family member, a hexokinase isoform or family member, or glutamine-fructose-6-phosphate transaminase.

10. The method of claim 9, wherein the glycogen targeting subunit of PP-1 family member comprises G_{L} (PPP1R3B, PPP1R4), PTG (PPP1R3C, PPP1R5), PPP1R3D (PPP1R6) or Gₘ/R_{G1} (PPP1R3A, PPP1R3).

11. The method of claim 1, wherein the gene product comprises an antisense polynucleotide, a small interfering RNA molecule, or a ribozyme that reduces expression of a glycogenolytic enzyme.

12. The method of claim 11, wherein the glycogenolytic enzyme comprises glycogen phosphorylase, debranching enzyme, phosphorylase kinase, glucose-6-phosphatase, PPP1R1A (protein phosphatase 1, regulatory Inhibitor subunit 1A), PPP1R2 (protein phosphatase 1, regulatory subunit 2), phosphofructokinase, a glycogen synthase kinase-3 isoform, GCKR glucokinase regulatory protein or α-glucosidase.

13. The method of claim 1, wherein the cell comprises a hyperproliferative cell.

14. The method of claim 13, wherein the hyperproliferative cell comprises a metastatic or non-metastatic cancer cell.

15. The method of claim 14, wherein the cancer cell is present in brain, head or neck, breast, esophagus, mouth, stomach, lung, gastrointestinal tract, liver, pancreas, kidney, adrenal gland, bladder, colon, rectum, prostate, uterus, cervix, ovary, testes, skin, muscle or hematopoetic system.

16. The method of claim 14, wherein the hyperproliferative cell is present in a subject.

17. (canceled)

18. The method of claim 16, wherein the subject is a human.

19. The method of claim 1, wherein the gene product comprises a protein, an antisense polynucleotide, a small interfering RNA or a ribozyme.

20. The method of claim 1, wherein the gene product is encoded by a polynucleotide.

21. The method of claim 20, wherein the polynucleotide comprises a vector.

22. The method of claim 20, wherein the vector comprises a viral or mammalian expression vector.

23. The method of claim 20, wherein the polynucleotide further comprises a vesicle.

24. The method of claim 1, wherein expression of the gene product is conferred by a promoter active in a hyperproliferative cell.

25. The method of claim 24, wherein the promoter comprises hexokinase II, COX-2, alpha-fetoprotein, carcinoembryonic antigen, DE3/MUC 1, prostate specific antigen, C-erB2/neu, telomerase reverse transcriptase or hypoxia-responsive promoter.

26. The method of claim 1, further comprising expressing in the cell a second protein that inhibits cell proliferation.

27. The method of claim 26, wherein the second protein comprises a cell cycle inhibitor.

28. The method of claim 26, wherein the second protein comprises a cyclin inhibitor.

29. A method of increasing glycogen to toxic levels in a hyperproliferative cell, comprising contacting the cell with an agent that increases the amount of glycogen to toxic levels in the hyperproliferative cell, wherein the hyperproliferative cell is not a liver, muscle or brain cell.

30. The method of claim 29, wherein the glycogen is in an amount that causes a morphological change associated with glycogen toxicity.

31. The method of claim 29, wherein the glycogen is in an amount that causes lysis or apoptosis of the cell.

32. The method of claim 29, wherein the glycogen is in an amount that inhibits or reduces proliferation, growth or survival of the cell.

33. The method of claim 29, wherein the agent increases expression or activity of a glycogenic enzyme.

34. The method of claim 33, wherein the glycogenic enzyme is selected from glycogenin, glycogenin-2, glycogen synthase, glycogenin interacting protein (GNIP), protein phosphatase 1 (PP-1), glucose transporter (GLUT), a glycogen targeting subunit of PP-1 isoform or family member, a hexokinase isoform or family member, or glutamine-fructose-6-phosphate transaminase.

35. The method of claim 29, wherein the agent decreases expression or activity of a glycogenolytic enzyme.

36. The method of claim 29, wherein the agent comprises an antisense, ribozyme, siRNA or triplex forming nucleic acid that specifically binds to a glycogenolytic enzyme.

37. The method of claim 35, wherein the glycogenolytic enzyme is selected from glycogen phosphorylase, debranching enzyme, phosphorylase kinase, glucose-6-phosphatase, PPP1R1A (protein phosphatase 1, regulatory Inhibitor subunit 1A), PPP1R2 (protein phosphatase 1, regulatory subunit 2), phosphofructokinase, a glycogen synthase kinase-3 isoform, GCKR glucokinase regulatory protein or α-glucosidase.

38. A method of increasing glycogen to toxic levels in a hyperproliferative cell, comprising contacting the cell with an agent that increases the amount of glycogen to toxic levels in the hyperproliferative cell, provided that the agent does not substantially inhibit activity or expression of a glycogen phosphorylase isotype.

39. The method of claim 38, wherein the glycogen phosphorylase isotype comprises a liver, muscle or brain glycogen phosphorylase.

40. The method of claim 38, wherein the glycogen is in an amount that causes a morphological change associated with glycogen toxicity.

41. The method of claim 38, wherein the glycogen is in an amount that causes lysis or apoptosis of the cell.

42. The method of claim 38, wherein the glycogen is in an amount that inhibits or reduces proliferation, growth or survival of the cell.

43. The method of claim 38, wherein the agent increases expression or activity of a glycogenic enzyme.

44. The method of claim 43, wherein the glycogenic enzyme is selected from glycogenin, glycogenin-2, glycogen synthase, glycogenin interacting protein (GNIP), protein phosphatase 1 (PP-1), glucose transporter (GLUT), a glycogen targeting subunit of PP-1 isoform or family member, a hexokinase isoform or family member, or glutamine-fructose-6-phosphate transaminase.

45. The method of claim 38, wherein the agent decreases expression or activity of a glycogenolytic enzyme.

46. The method of claim 38, wherein the agent comprises an antisense, ribozyme, sIRNA or triplex forming nucleic acid that specifically binds to a glycogenolytic enzyme.

47. The method of claim 45, wherein the glycogenolytic enzyme is selected from debranching enzyme, phosphorylase kinase, glucose-6-phosphatase, PPP1R1A (protein phosphatase 1, regulatory Inhibitor subunit 1A), PPP1R2 (protein phosphatase 1, regulatory subunit 2), phosphofructokinase, a glycogen synthase kinase-3 isoform, GCKR glucokinase regulatory protein or α-glucosidase.

48. The method of claim 38, wherein the agent comprises a substrate analogue.

49. The method of claim 38, wherein the hyperproliferative cell comprises a metastatic or non-metastatic cancer cell.

50. The method of claim 49, wherein the cancer cell is present in brain, head or neck, breast, esophagus, mouth, stomach, lung, gastrointestinal tract, liver, pancreas, kidney, adrenal gland, bladder, colon, rectum, prostate, uterus, cervix, ovary, testes, skin or muscle, or hematopoetic system.

51. The method of claim 38, wherein the hyperproliferative cell is present in a subject.

52. (canceled)

53. The method of claim 51, wherein the subject is a human.

54. A method of treating a cell proliferative disorder in a subject, wherein the cell proliferative disorder is not a liver, muscle or brain cell disorder, comprising expressing in one or more cells comprising the disorder a gene product that increases the amount of intracellular glycogen, or comprising contacting one or more cells comprising the disorder with an agent that increases the amount of intracellular glycogen, sufficient to treat the cell proliferative disorder.

55. The method of claim 54, wherein the cell proliferative disorder comprises a metastatic or non-metastatic cancer.

56. The method of claim 55, wherein the cancer cell is present in head or neck, breast, esophagus, mouth, stomach, lung, gastrointestinal tract, pancreas, kidney, adrenal gland, bladder, colon, rectum, prostate, uterus, cervix, ovary, testes, skin, or hematopoetic system.

57. A method of treating a cell proliferative disorder of a subject, comprising expressing in one or more cells comprising the disorder a gene product that increases the amount of intracellular glycogen, or comprising contacting one or more cells comprising the disorder with an agent in an amount that increases the amount of intracellular glycogen, provided that the agent does not substantially inhibit activity or expression of a glycogen phosphorylase isotype, sufficient to treat the cell proliferative disorder.

58. The method of claim 57, wherein the cell proliferative disorder comprises a metastatic or non-metastatic cancer.

59. The method of claim 58, wherein the cancer cell is present in brain, head or neck, breast, esophagus, mouth, stomach, lung, gastrointestinal tract, liver, pancreas, kidney, adrenal gland, bladder, colon, rectum, prostate, uterus, cervix, ovary, testes, skin or muscle, or hematopoetic system.

60. (canceled)

61. The method of claims 54 and 57, wherein the subject is human.

62. A method of treating a subject having a tumor, wherein the tumor is not a liver, muscle or brain tumor, comprising expressing in one or more of the tumor cells a gene product that increases the amount of intracellular glycogen, or comprising contacting one or more of the tumor cells with an agent that increases the amount of intracellular glycogen, effective to treat the subject.

63. A method of treating a subject having a tumor, comprising expressing in one or more of the tumor cells a gene product that increases the amount of intracellular glycogen, or comprising contacting one or more of the tumor cells with an agent in an amount that increases the amount of intracellular glycogen, provided that the agent does not substantially inhibit activity or expression of a glycogen phosphorylase isotype, effective to treat the subject.

64. A method of treating a subject that is undergoing or has undergone tumor therapy, wherein the tumor therapy was not for a liver, muscle or brain tumor, comprising administering to the subject an agent in an amount that increases the amount of intracellular glycogen in a cell sufficient to treat the subject.

65. A method of treating a subject that is undergoing or has undergone tumor therapy, comprising administering to the subject an agent in an amount that increases the amount of intracellular glycogen, provided that the agent does not substantially inhibit activity or expression of a glycogen phosphorylase isotype, sufficient to treat the subject.

66. A method of increasing effectiveness of an anti-tumor therapy, comprising administering to a subject that is undergoing or has undergone anti-tumor or immune-enhancing therapy, wherein the tumor therapy was not for a liver, muscle or brain tumor, an agent in an amount that increases the amount of intracellular glycogen, and an anti-tumor or immune-enhancing therapy.

67. A method of increasing effectiveness of an anti-tumor therapy, comprising administering to a subject that is undergoing or has undergone anti-tumor or immune-enhancing therapy, an agent in an amount that increases the amount of intracellular glycogen, provided that the agent does not substantially inhibit activity or expression of a glycogen phosphorylase isotype, and an anti-tumor or immune-enhancing therapy.

68. The method of any of claims 66 or 67, wherein the agent is administered prior to, substantially contemporaneously with or following administration of the anti-tumor or immune-enhancing therapy.

69. The method of any of claims 62 to 67, wherein the tumor comprises a metastatic or non-metastatic tumor.

70. The method of any of claims 62 to 67, wherein the tumor comprises a stage I, II, III, IV or V tumor.

71. The method of any of claims 62 to 67, wherein the tumor is in remission.

72. The method of any of claims 62 to 67, wherein the tumor is solid or liquid.

73. The method of any of claims 62, 64 and 66, wherein the tumor is located at least in part in head or neck, breast, esophagus, mouth, stomach, lung, gastrointestinal tract, pancreas, kidney, adrenal gland, bladder, colon, rectum, prostate, uterus, cervix, ovary, testes, or skin.

74. The method of any of claims 63, 65 and 67, wherein the tumor is located at least in part in brain, head or neck, breast, esophagus, mouth, stomach, lung, gastrointestinal tract, liver, pancreas, kidney, adrenal gland, bladder, colon, rectum, prostate, uterus, cervix, ovary, testes, skin or muscle.

75. The method of any of claims 62 to 67, wherein the tumor is haematopoetic.

76. The method of any of claims 62 to 67, wherein the tumor comprises a sarcoma, carcinoma, melanoma, myeloma, blastoma, glioma, lymphoma or leukemia.

77. The method of any of claims 62 to 67, wherein the treatment reduces tumor volume, inhibits an increase in tumor volume, inhibits progression of the tumor, stimulates tumor cell lysis or apoptosis, or inhibits tumor metastasis.

78. The method of any of claims 62 to 67, wherein the treatment reduces one or more adverse symptoms associated with the tumor.

79. The method of any of claims 62 to 67, wherein the treatment prolongs lifespan of the subject.

80. The method of any of claims 62 to 67, wherein the subject is a candidate for, is undergoing, or has undergone anti-tumor or immune-enhancing therapy.

81. The method of any of claims 62 to 67, further comprising administering an anti-tumor or immune enhancing treatment or agent.

82. The method of claim 81, wherein the anti-tumor treatment comprises chemotherapy, immunotherapy, surgical resection, radiotherapy or hyperthermia.

83. The method of claim 81, wherein the anti-tumor agent comprises an alkylating agent, anti-metabolite, plant extract, plant alkaloid, nitrosourea, hormone, nucleoside or nucleotide analogue.

84. The method of claim 81, wherein the anti-tumor agent is selected from: cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin and dibromomannitol.

85. The method of claim 81, wherein the immune enhancing treatment comprises administration of a lymphocyte, plasma cell, macrophage, dendritic cell, NK cell or B-cell.

86. The method of claim 81, wherein the immune enhancing agent comprises an antibody, a cell growth factor, a cell survival factor, a cell differentiative factor, a cytokine or a chemokine.

87. The method of claim 81, wherein the immune enhancing agent is selected from: IL-2, IL-1α, IL-1β, IL-3, IL-6, IL-7, granulocyte-macrophage-colony stimulating factor (GMCSF), IFN-γ, IL-12, TNF-α, TNFβ, MIP-1 α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROα, GROβ, ENA-78, GCP-2, PBP/CTAPIIIβ-TG/NAP-2, Mig, PBSF/SDF-1, and lymphotactin.

88.-126. (canceled)


SEQUENCE LISTING (9 sequences)

SEQ ID NO: 1 - DNA, 26 bp, Artificial Sequence
  Description of Artificial Sequence sense primer
gaccaattgtcgcgcttgccacaacc

SEQ ID NO: 2 - DNA, 25 bp, Artificial Sequence
  Description of Artificial Sequence anti-sense primer
ctgctcgagcgcgccagccaccact

SEQ ID NO: 3 - DNA, 3807 bp, Homo sapiens
gacatctccgggaacccagcccaggccctgcctcccggacacaccgacgctcacgtagtcgcgcttgccacaaccctgcgggctctccgatgcggcgagcgagctggggagggggcttctccgcggcccaaaagcctgttcatctagccccatgatggctgtggacatcgagtacagatacaactgcatggctccttccttgcgccaagagaggtttgcctttaagatctcaccaaagcccagcaaaccactgaggccttgtattcagctgagcagcaagaatgaagccagtggaatggtggccccggctgtccaggagaagaaggtgaaaaagcgggtgtccttcgcagacaaccaggggctggccctgacaatggtcaaagtgttctcggaattcgatgacccgctagatatgccattcaacatcaccgagctcctagacaacattgtgagcttgacgacagcagagagcgagagctttgttctggatttttcccagccctctgcagattacttagactttagaaatcgacttcaggccgaccacgtctgccttgagaactgtgtgctcaaggacaaggccattgcaggcactgcgaaggttcagaacctcgcatttgagaagaccgtgaaaataaggatgacgttcgacacctggaagagctacacagactttccttgtcagtacgtgaaggacacttatgccggttcagacagggacacgttctccttcgacatcagtttgcccgagaagattcagtcttatgaaagaatggagtttgctgtgtactacgagtgcaatggacagacgtactgggacagcaacagaggcaagaactataggatcatccgggctgagttaaaatctacccagggaatgaccaagccccacagtggaccggatttgggaatatcctttgaccagttcggaagccctcggtgttcctatggtctgtttccagagtggccaagttacttaggatatgaaaagctagggccctactactagtgactgcaggtgacagggcgtggcggagctgccacagacaagcctagctctgctcactgtgcagtggagatggaaggccagggaggagcaacgtggaacttccatgaggccccgtttgggaaaataaaaggatcctcttcacttctttcttaaacagcaaatccagccaggttcagattacacaaccagtgtctcactcaaaggagcagtggtggctggcgcgctttcgcactgtggcagccacgagagtctgtgcacgtctgtgctggaaagggtatggatgggaatcaagcctatgccagtgctgatgaagctggaggagtctctcttctgctctccactcagatgtgggacatcagtcgccaaaagccactcagccccagccacctcgcgtgagaccctcactgttcattgtgttcatctttgggtgctctctgccagccaggcctttcctgcaagctgctgtgcttccccgtccacgtgtatctctgctgtgacacactgagctgacgcacatctccagtgcagctgcagaagagaaatgggattggctcttgttttctgcaagttcatgttttgcattttatgttcttccacaattgatctgatgttcaggaaaagataataaaggcaaattagttagtggttgagacaggcatttcctcctcccgcttcttgaccccacagatgtattccagcagagagcaacacaccagtcatcaaaacccactggctcctgtgcggtgtcacagattgcagggttctgacaaggcaggacagtcaagagtggggacactttcagcttctacttttgccttctagggggagctttctaagtccccacatttaccccgagtcaccggaaaaatctgatttttcccccgaaagctcaatgactttaacgtgcttggctggtttgtctcattctttatgaaagaattttggggccgggcgcggtggcttatgcctgtaatcccagcactttgggaggccgaggcaggtggatcacgaggtcaggagatcgagaccatcctggctaacacggtgaaaccctgtctctactaaaaatacaaaaaaattagccaggcgtggtggcgggcgcctgtagtcccagctacttgggaggctgaggcaggagaatggcgggaacctgggaggcggagcttacagtgaaccgagatcacaccactgcattcccgcctgggcagcagagcgagactccgtctcaaacagaaaaaaaaagagaattttgaatctcctttcccaaagagtcatcttttctgctgtgtttaggacatttgatttgcatatccaatatctcctcgaaaccttcagaaaatggttttatcgtgactgtgattcacactatctagaacactttaccagcacccagggacatggacttgggtgttcttatttatggtgtgtatgtaaagagataggggagaaaaacctcacccaagttcttatacgttattttaaacgtttgccaactctgaaatttcagagattcattgttcttaaccatattggactaaagtctgttggttagtgctgttgtaaaagagacctctggggccgagtgtggtggcttatgcctgtaatccctgcactttgggaagccaaggcgggcagctcacctgaggtcaggcgttcaagaccagcctggccaacatggtgaaacctcgtctgtactaaaaatacaaaatttagctgagtgtggtggcaggcgcctgtaatccagctactcgggaggctgaggcaggagaatcacttgaacccaggaggtggaggttgcagtgagccgagatcacgtcactgcatttcagcctgagcgacagagtgaaactgtctcaaaaaagagagacttccgggacagtcattatcagataggccccaaacctgtgatttttcttgggcaagattggtgtttacttaggggtgttttaaaaatatatttttacaagtatacttgtaggaagttggttttttattttctatttttgtttgtttttggagtttggttaaacgactcttttattttctgttttgccttatcttactaaagtgaagttttcctaaggcaagcaagaagaggagtggaagcacagttgcctggatttggaggcagagttgtcaggcttttcaagctaagagtcttgtgcttggattttccagattaatttgaaaagacctcccatttgtggctttgtacataaccaacaggcagatactgagtgccttggctcctagagttttgtggttgggttaggttgtttttgttgttgttttgttttggttttgttttcatttctgaagtttaagatgccttgactttttaaatgctcttaaggctattcgatgtaattcttactcctaaaactggctgttcttagcctgaaatctaatgctttgttttttgtacctctcccaggtgggtaacactctaggatgacatgatgttataattttaggggaaatcacatttttaccttatgctgttactgggcagaaccacgttttatgtaaacataccagacatcgggtaacagacagtactttaaatgttataaatttggtgatcagaactattaatagcataaatcgaactcaaatggaagcaaaactgatttcatgcaggtcctgaattttactttgccttaagaagtgccctccccacaatgcaggagaggcacagagtgcactgtcattgacatgttacccgactaaggatcactctgttcataagaaaaaggcctgaagtgactctgtttaataaagtcagtttaattttatct

SEQ ID NO: 4 - DNA, 30 bp, Artificial Sequence
  Description of Artificial Sequence sense primer
ggcaattgaacggctagcctgaggagctgc

SEQ ID NO: 5 - DNA, 29 bp, Artificial Sequence
  Description of Artificial Sequence anti-sense primer
ccactagtgcggttccctgctctctgtcg

SEQ ID NO: 6 - DNA, 2691 bp, Homo sapiens
cgccatggagaccaacacccttcccaccgccactcccccttcctctcagggtccctgtcccctccagtgaatcccagaagactctggagagttctgagcagggggcggcactctggcctctgattggtccaaggaaggctggggggcaggacgggaggcgaaacccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagccgagccgacagagagcagggaaccgcatggccaaagccgcggcagtcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcgcctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtcctgatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtgggctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggccccaggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattaggggcctttccaagattgctgtttttgttttggagcttcaagactttgcatttcctagtatttctgtttgtcagttctcaatttcctgtgtttgcaatgttgaaattttttggtgaagtactgaacttgcctttttttccggtttctacatgcagagatgaatttatactgccatcttacgactatttcttctttttaatacacttaactcaggccattttttaagttggttacttcaaagtaaataaactttaaaattcaagtgatgcccttttattcct

SEQ ID NO: 7 - DNA, 28 bp, Artificial Sequence
  Description of Artificial Sequence sense primer
tcgggatccaatcaacctctggattaca

SEQ ID NO: 8 - DNA, 24 bp, Artificial Sequence
  Description of Artificial Sequence anti-sense primer
tgctctagacaagcaacacggacc

SEQ ID NO: 9 - DNA, 3308 bp, Marmota monax
  misc_feature: Woodchuck hepatitis B virus
aattcgggacataccacgtggtttagttccgcctcaaactccaacaaatcgagatcaagggagaaagcctactcctccaactccacctctaagagatactcacccccacttaactatgaaaaatcagacttttcatctccaggggttcgtggacggattaagagacttgacaacaacggaacgccaacacaatgcctatggagatccttttacgacactaagccctgtggttcctactgtatccaccatattgtctcctccatcgacgactggggaccctgcacagtcaccggagatgtcaccatcaagtctcctaggactcctcgcaggattacaggtggtgtatttcttgtggacaaaaatcctaacaatagctcagaatctagattggtggtggacttctctcagttttccagggggcataccagagtgcactggccaaaattcgcagttccaaacttgcaaacacttgccaacctcctgtccaccgacttgcaatggctttcgttggatgtatctgcggcgttttatcatatacctattagtcctgctgctgtgcctcatcttcttgttggttctcctggactggaaaggtttaatacctgtctgtcctattcaacccacaacagaaacgacagtcaattgcagacaatgcacaatctctgtacaagacatgtatactcctccttactgttgttgtttaaaacctacggcaggaaattgcacttgctggcccatcccttcatcatgggctttaggaaattacctatgggagtgggccttagcccgtttctcttggctcaatttactagtgcccttgcttcaatggttaggaggaatttccctcattgcgtggtttttgcttatatggatgatttggttttgggggcccgcacttctgagcatcttaccgccatttatacccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagcaaggacctttggactccttatataagagatcaattattaactaaatgggaggagggcagcattgatcctagattatcaatatttgtattaggaggctgtaggcataaatgcatgcgacttccgtaaccatgtatctttttcacctgtgccttgtttttgcctgtgttccatgtcctactgttcaagcctccaagctgtgccttggatggctttggggcatggacatagatccttataaagaatttggttcatcttatcagttgttgaattttcttcctttggacttctttcctgatcttaatgctttggtggacactgctactgccttgtatgaagaagaactaacaggtagggaacattgctctccgcaccatacagctattagacaagctttagtatgctgggatgaattaactaaattgatagcttggatgagctctaacataacttctgaacaagtaagaacaatcattgtaaatcatgtcaatgatacctggggacttaaggtgagacaaagtttatggtttcatttgtcatgtctcactttcggacaacatacagttcaagaatttttagtaagttttggagtatggatcaggactccagctccatatagacctcctaatgcacccattctctcgactcttccggaacatacagtcattaggagaagaggaggtgcaagagcttctaggtcccccagaagacgcactccctctcctcgcaggagaagatctcaatcaccgcgtcgcagacgctctcaatctccatctgccaactgctgatcttcaatgggtacataaaactaatgctattacaggtctttactctaaccaagctgctcagtttaatccgcattggattcaacctgagtttcctgagcttcatttacacaatgaattaattaaaaaattgcaacagtattttggtcccctgactataaatgaaaagagaaaattgcaattaaattttcctgcaagatttttccccaaagccactaaatattttcctttaattaaaggcataaaaaacaattatcctaattttgctttagaacatttctttgctaccgcaaattatttgtggactttatgggaagctggaattttgtatttaaggaagaatcaaacaactttgacttttaaaggtaaaccatattcttgggaacacagacagctagtgcaacataatgggcaacaacataaaagtcaccttcaatccagacaaaatagcagcgtggtggcctgcagtgggcacttattacacaaccacttaccctcagaaccagtcagtgtttcaaccaggaatttatcaaacaacatctttggtaaatcccaaaactcaacaagaactggactctgttctcataaacagatacaaacagatagattggaacacttggcaaggatttcctgtcgatcaaaaactaccattggtcaacagggatcctcccccaaaatcagctcaaactttcgaaatcaaacctgggcctataatagttcctgg